(19) United States
(12) Patent Application Publication
(10) Pub. No.: US 2019/0175766 A1
(43) Pub. Date: Jun. 13, 2019

(54) STABILIZED CROSSLINKED NANOBUBBLES FOR DIAGNOSTIC AND THERAPEUTIC APPLICATIONS

(71) Applicant: CASE WESTERN RESERVE UNIVERSITY, Cleveland, OH (US)

(72) Inventors: Agata Exner, Westlake, OH (US); Reshani Perera, Mayfield Heights, OH (US); Chris Hernandez, Providence, RI (US)

(21) Appl. No.: 16/306,840

(22) PCT Filed: Jun. 2, 2017
(86) PCT No.: PCT/US17/35766

§ 371 Date: Dec. 3, 2018

Related U.S. Application Data
(60) Provisional application No. 62/344,551, filed on Jun. 2, 2016.

(51) Int. Cl. A61K 49/22; A61K 9/51; A61K 41/00; A61K 9/107; A61K 9/00; A61K 31/704; A61K 47/69; A61K 49/00

(52) U.S. Cl. CPC A61K 49/223; A61K 9/5138; A61K 41/0033; A61K 9/1075; A61K 49/0093; A61K 31/704; A61K 47/6925; A61K 9/5146; A61K 9/0019

(57) ABSTRACT

A composition includes a plurality of stabilized crosslinked nanobubbles, each nanobubble having a membrane that defines at least one internal void, which includes at least one gas, the membrane including at least one lipid, at least one nonionic triblock copolymer that is effective to control the size of the nanobubble without compromising in vitro and in vivo echogenicity of the nanobubble, and an interpenetrating crosslinked biodegradable polymer.

### RELATED APPLICATION

This application claims priority from U.S. Provisional Application No. 62/344,551, filed Jun. 2, 2016, the subject matter of which is incorporated herein by reference in its entirety.

### GOVERNMENT FUNDING

This invention was made with government support under Grant No. W81XWH-12-1-0500 awarded by The Department of Defense, Advanced Research Projects Agency. The United States government has certain rights to the invention.

### TECHNICAL FIELD

This application relates to diagnostic and therapeutic compositions, and more particularly to stabilized crosslinked nanobubbles for diagnostic, therapeutic, and theranostic applications.

### BACKGROUND

Ultrasound contrast agents (UCA) are small gas-filled bubbles with a stabilizing shell made from a variety of materials, such as polymer, protein or lipid. Other than the traditional applications of these agents in diagnostic ultrasound imaging, UCA have found relevance in therapeutic applications including targeted gene and drug delivery. These adaptable particles are currently being explored as protective therapeutic carriers and as cavitation nuclei to enhance delivery of their payload by sonoporation. Together these functions improve payload circulation half-life and release profiles as well as tissue selectivity and cell uptake. Regardless of the mode of action, it is advantageous, particularly in cancer therapy, for the bubbles to extravasate from the vasculature and arrive at the cellular target site for the desired effect.

Commercial UCA available today are typically designed to serve only as blood pool agents with diameters of 1-8 μm. Although previous methodologies have been developed to reduce bubble size, most of these strategies involve manipulations of microbubbles post formation, such as gradient separation by gravitational forces or by physical filtration or floatation. While effective for selecting nanosized bubbles, these methods introduce potential for sample contamination, reduce bubble yield and stability, and waste stock materials in addition to being labor intensive. Additionally, the applicability of microbubbles as carriers (e.g., in cancer therapy) has been limited by a large size, which typically confines them to the vasculature.

### SUMMARY

Embodiments described herein relate to stabilized crosslinked nanobubbles for diagnostic and therapeutic applications. The stabilized crosslinked nanobubbles can be used as multifunctional and/or theranostic platforms for molecular imaging, drug therapy, gene therapy, chemotherapy, and anti-microbial applications. The stabilized crosslinked nanobubble can include a membrane that defines an internal void. The internal void can include at least one gas. The membrane can include at least one lipid, at least one nonionic triblock copolymer that is effective to control the size of the nanobubble without compromising in vitro and in vivo echogenicity of the nanobubble, and an interpenetrating crosslinked biodegradable polymer.

Advantageously, the stabilized crosslinked nanobubbles can have a substantially smaller diameter and a significant improvement in stability and retention of echogenic signal over 24 hours compared to similar stabilized nanobubbles that are not crosslinked. Moreover, in vivo analysis via ultrasound and fluorescence mediated tomography showed greater tumor extravasation and accumulation of the stabilized crosslinked nanobubbles compared to microbubbles.

In some embodiments, the nanobubble can include a hydrophilic outer domain at least partially defined by hydrophilic heads of the lipid and the nonionic triblock copolymer and a hydrophobic inner domain at least partially defined by hydrophobic tails of the lipid. The interpenetrating cross-linked biodegradable polymer can be non-covalently integrated into the hydrophobic domain of the nanobubble.

In other embodiments, the nonionic triblock copolymer can include at least one poloxamer. The poloxamer can have a molecular weight, for example, of about 1100 Daltons to about 3500 Daltons. The concentration of nonionic triblock copolymer in the lipid nanobubble can be about 0.06 mg/ml to about 1 mg/ml. The poloxamer:lipid molar ration can be about 0.02 to about 0.5, for example, about 0.1 to about 0.3. The gas can have a low solubility in water and include, for example, a perfluorocarbon, such as perfluoropropane, carbon dioxide, and air.

In another aspect, the nanobubble can have a size that facilitates extravasation of the nanobubble in cancer therapy or diagnosis. For example, the nanobubble can have a diameter or size of about 30 nm to about 300 nm (or about 50 nm to about 200 nm or about 50 nm to about 150 nm).

In a further aspect, the nanobubble can include at least one targeting moiety that is linked to the membrane. The targeting moiety can be selected from the group consisting of polypeptides, polynucleotides, small molecules, elemental compounds, antibodies, and antibody fragments.

In a still further aspect, the nanobubble can include at least one therapeutic agent that is contained within the membrane or conjugated to the membrane. The therapeutic agent can include at least one chemotherapeutic agent, anti-proliferative agent, biocidal agent, biostatic agent, or anti-microbial agent.

Other embodiments described herein relate to a method for forming a composition comprising at least one stabilized nanobubble. The method can include the steps of: (a) dissolving at least one lipid in a solvent; (b) evaporating the solvent to produce a film; (c) hydrating the film; and (d) removing air, injecting gas and shaking a solution of the hydrated film to form the at least one nanobubble. At least one nonionic triblock copolymer, acrylamide monomer, crosslinking agent (or crosslinker), and crosslinking initiator, can be added at either step (a) or step (c) to control the size and stability of the at least one nanobubble.

A further aspect of the application relates to a method for imaging a region of interest (ROI) in a subject. The method can include administering to the subject a composition comprising a plurality of stabilized nanobubbles. Each of the nanobubbles can have a membrane that defines an internal void. The internal void can include at least one gas. The membrane can include at least one lipid, at least one nonionic triblock copolymer that is effective to control the size of the nanobubble without compromising in vitro and in vivo echogenicity of the nanobubble, and an interpenetrating cross-linked biodegradable polymer. After administering the composition to the subject, at least one image of the ROI can be generated.

A further aspect of the application relates to a method for treating a neoplastic disorder in a subject. The method can include administering to neoplastic cells of the subject a composition comprising a plurality of stabilized nanobubbles. Each of the nanobubbles can have a membrane that defines an internal void. The internal void can include at least one gas. The membrane can include at least one lipid, at least one nonionic triblock copolymer that is effective to control the size of the nanobubble without compromising in vitro and in vivo echogenicity of the nanobubble, and an interpenetrating cross-linked biodegradable polymer. A chemotherapeutic agent can be provided on or in the nanobubble.

Ultrasound can then be applied to a region of interest of the subject that includes the neoplastic cells and nanobubbles to cause release of the chemotherapeutic agent from the nanobubbles in the region of interest to the neoplastic cells.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a schematic diagram of lipid and Pluronic-stabilized nanobubbles with interpenetrating crosslinking biodegradable polymer N, N-diethyl acrylamide (NNDEA) and N,N-bis(acryoyl) cystamine (BAC) crosslinking network.

FIG. 2 is a schematic diagram of a method of forming the stabilized nanobubble.

FIG. 3 is a schematic diagram of stabilized crosslinked drug loaded nanobubble in accordance with another embodiment.

FIGS. 4(A-E) illustrates size distribution of the CL-PEG-NB obtained by (A) DLS and (B) qNano; (C) Concentration distribution of CL-PEG-NB obtained from qNano; (D) STED image of Alexa Fluor 488 tagged CL-EG-NB; (E) Surface morphology of the CL-PEG-NB and normal PEG-NB visualized using scanning electron microscopy (SEM).

FIGS. 5(A-B) illustrate (A) Representative ultrasound images of nanobubbles in vitro, showing both custom-made tissue phantom area and the contrast agent area (B) Enhanced in vitro stability of CL-PEG-NB compared to original PEG-NB over 1 h (inset) and 24 h as measured as a function of time.

FIG. 6 illustrates ultrasound images showing the contrast in each organ and the tumor after the bubble injection as a function of time. Regions of interests (ROIs) delineate the liver, kidney, and subcutaneous tumor.

FIGS. 7(A-C) illustrate ultrasound intensity of each organ and the tumor after injecting (A) PEG-NB; (B) CL-PEG-NB; (C) Normalized ultrasound intensity of each organ at the initial phase after peak enhancement (n=3).

FIGS. 8(A-B) illustrate tumor nanobubble and microbubble kinetics measured with FMT. (A) FMT images show the accumulation of Vivo680-tagged CL-PEG-NB and Vivo680 tagged PEG-MB in tumor at 1 h post injection; (B) Fluorescence intensities of tumor after application of above treatments as a function of time. In each case, CL-PEG-NB perform better than the control.

FIG. 9 illustrate fluorescence intensities of each organ (liver, kidney, heart) 5 and 60 min of post application of Vivo680-CL-PEG-NB and Vivo680-PEG-MB.

FIGS. 10(A-C) illustrate (A) Representative fluorescence images showing the bubble distribution in tumor. Alexa 488-tagged CL-PEG-NB showed higher extravasation compared to the Alexa 488-tagged PEG-MB. MB confined to the tumor vasculature (red); (B) Representative image showing the extravasation of CL-PEG-NB into the tumor beyond the tumor vasculature; (C) The signal intensities of bubble and vessel expressed as the percentage of total cells of tumor tissues.

FIGS. 11(A-C) illustrate change in nanobubble (A) US signal, (B) size and (C) shape (by cryo-EM) before and after destruction with high intensity US. Color added to cryoEM images for better visualization. Scale bars represent 100 nm.

FIGS. 12(A-C) illustrate the effect of lipid acyl chain length on nanobubble stability under ultrasound exposure. (A) Schematic of acrylamide phantom used to measure in vitro stability. (B) Typical ultrasound images of nanobubbles at different time points.

(C) Representative ultrasound time intensity curves and average half-life of each formulation.

FIGS. 13(A-D) illustrate (A) Schematic of animal tumor model and US scan orientation. (B) MIP comparison in PC3 flank tumor 15 s after contrast injection.

(C) Representative tumor images of NBs and MBs from the same mouse. (D) Mean TIC curves for NBs and MBs.

FIG. 14 is a structural representation of Dox-NB Pluronic lipids membrane and stabilizing mesh with Dox particles and C₃F₈ gas inside.

FIGS. 15(A-D) illustrate (A-C) Fluorescence microscopy images showing Dox-NB with red dot in rhodamine channel (63×). D. The diameters of Dox-NBs are 100-300 nm (mean value: 171.5 nm) by DSL measurement.

FIG. 16 illustrates cell toxicity test of Dox-NB for LS174T cell line. Dox-NB+TUS had much more toxic effect in vitro compared to other groups (p<0.05).

FIGS. 17(A-E) illustrate fluorescence microscope images of LS174T cell line in different treatments. A-E Confocal fluorescence microscopy images (20×) for each group (Rhodamine channel, 800 ms expose). The value of fluorescence intensity calculated by ImageJ are shown in right lower corner. Dox-NB+TUS treatment got significant higher Dox fluorescence compared to other groups.

FIG. 18 illustrates Dox amount in tumors of different treatment. Tissue homogenates analysis shows that the concentration of Dox was 1.25 μg per 1 g tumor tissue in Dox-NB+TUS group, which was significantly higher than that in other groups (TECAN analysis).

FIGS. 19(A-B) illustrate Maestro images for mice tumors of 3 h treatment. Tumor tissue, heart, liver and kidney images on Maestro image system with 1000 ms expose (Extraction, blue filter; Emission, green filter). Unmix pictures analysis of the tumor areas in different groups shows that the value of fluorescence intensity for the tumor area in Dox-NB+TUS group was higher than other groups but not statistically significant.

FIGS. 20(A-D) illustrate fluorescence microscope images of mice tumors histology. Confocal fluorescence microscopy images (20×) of mice tumors in different treatment groups (5 mice/group); the blue color indicated the nuclei stained with DAPI and the red color indicated presence of Dox. The fluorescence ratio (Dox/DAPI) was compared between Dox-NB+TUS group and other groups, Dox-NB+TUS group has significant higher fluorescence ratio (Dox/DAPI) than other groups (p<0.0001).

FIGS. 21(A-B) illustrate relative tumor volume and survive test of different treatment groups. A. Relative tumor volume changes Compared to Dox group, Dox-NB+TUS significantly decreased tumor growth (p<0.05). B. Kaplan-Meier survival analysis shows Dox-NB+TUS treatment significantly improved the overall survival of the colon tumor-bearing mice compared to control group and Dox group.

### DETAILED DESCRIPTION

All scientific and technical terms used in this application have meanings commonly used in the art unless otherwise specified. The definitions provided herein are to facilitate understanding of certain terms used frequently herein and are not meant to limit the scope of the application.

As used herein, the term “neoplastic disorder” can refer to a disease state in a subject in which there are cells and/or tissues which proliferate abnormally. Neoplastic disorders can include, but are not limited to, cancers, sarcomas, tumors, leukemias, lymphomas, and the like.

As used herein, the term “neoplastic cell” can refer to a cell that shows aberrant cell growth, such as increased, uncontrolled cell growth. A neoplastic cell can be a hyperplastic cell, a cell from a cell line that shows a lack of contact inhibition when grown in vitro, a tumor cell, or a cancer cell that is capable of metastasis in vivo. Alternatively, a neoplastic cell can be termed a “cancer cell.” Non-limiting examples of cancer cells can include melanoma, breast cancer, ovarian cancer, prostate cancer, sarcoma, leukemic retinoblastoma, hepatoma, myeloma, glioma, mesothelioma, carcinoma, leukemia, lymphoma, Hodgkin lymphoma, Non-Hodgkin lymphoma, promyelocytic leukemia, lymphoblastoma, thymoma, lymphoma cells, melanoma cells, sarcoma cells, leukemia cells, retinoblastoma cells, hepatoma cells, myeloma cells, glioma cells, mesothelioma cells, and carcinoma cells.

As used herein, the term “tumor” can refer to an abnormal mass or population of cells that result from excessive cell division, whether malignant or benign, and all pre-cancerous and cancerous cells and tissues.

As used herein, the terms “treating” or “treatment” of a disease (e.g., a neoplastic disorder) can refer to executing a treatment protocol to eradicate at least one neoplastic cell. Thus, “treating” or “treatment” does not require complete eradication of neoplastic cells.

As used herein, the term “polymer” can refer to a molecule formed by the chemical union of two or more chemical units. The chemical units may be linked together by covalent linkages. The two or more combining units in a polymer can be all the same, in which case the polymer may be referred to as a homopolymer. The chemical units can also be different and, thus, a polymer may be a combination of the different units. Such polymers may be referred to as copolymers.

As used herein, the term “block copolymer” can refer to a polymer in which adjacent polymer segments or blocks are different, i.e., each block comprises a unit derived from a different characteristic species of monomer or has a different composition of units.

As used herein, the term “poloxamer” can refer to a series of non-ionic triblock copolymers comprised of ethylene oxide and propylene oxide. Poloxamers are synthesized by the sequential addition of propylene oxide, followed by ethylene oxide, to propylene glycol. The poly(oxyethylene) segment is hydrophilic and the poly(oxypropylene) segment is hydrophobic. The molecular weight of poloxamers may range from 1000 to greater than 16000. The basic structure of a poloxamer is HO—(C₂H₄O)ₐ(C₃H₆O)_{b}(C₂H₄O)ₐ—H, where “a” and “b” represent repeating units of ethylene oxide and propylene oxide, respectively.

As used herein, the term “poloxamine” can refer to a polyalkoxylated symmetrical block copolymer prepared from an ethylene diamine initiator. Poloxamines are synthesized using the same sequential order of addition of alkylene oxides as used to synthesize poloxamers. Structurally, the poloxamines include four alkylene oxide chains and two tertiary nitrogen atoms, at least one of which is capable of forming a quaternary salt. Poloxamines are also terminated by primary hydroxyl groups.

As used herein, the term “meroxapol” can refer to a symmetrical block copolymer consisting of a core of polyethylene glycol (PEG) polyoxypropylated to both its terminal hydroxyl groups, i.e., conforming to the general type (PPG)ₓ-(PEG)_{y}-(PPG)ₓ, wherein “x” and “y” represent repeating units of PPG and PEG, respectively, and being formed by an ethylene glycol initiator. As opposed to the poloxamers, which are terminated by two primary hydroxyl groups, meroxapols have secondary hydroxyl groups at the ends and the hydrophobe is split in two, each half on the outside of the surfactant.

As used herein, the term “subject” can refer to any animal, including, but not limited to, humans and non-human animals (e.g., rodents, arthropods, insects, fish (e.g., zebrafish)), non-human primates, ovines, bovines, ruminants, lagomorphs, porcines, caprines, equines, canines, felines, ayes, etc.), which is to be the recipient of a particular treatment.

Embodiments described herein relate to stabilized crosslinked nanobubbles for diagnostic, therapeutic, and/or theranostic applications. The stabilized crosslinked nanobubbles can be used as multifunctional platforms for molecular imaging, drug therapy, gene therapy, chemotherapy, and/or anti-microbial applications. Nonionic triblock copolymers (e.g., poloxamers) when combined with lipids and cross-linking agents can form stabilized crosslinked nanobubble contrast agents that when administered to a subject with cancer are clearly visible on ultrasound yet sufficiently small to move beyond leaky tumor vasculature, permitting greatly expanding molecular imaging capabilities of ultrasound at clinically relevant frequencies (e.g., 1 to 20 MHz).

The membranes of the stabilized crosslinked nanobubbles described herein are tightly packed permitting a smaller size than traditionally formed microbubbles. Particle diameter has been the most widely accepted factor, which governs the resonant frequency of the bubble and its visibility with ultrasound. Typically, smaller bubbles vibrate faster, making them extremely difficult to detect with clinically relevant ultrasound frequencies. The stabilized crosslinked nanobubbles described herein, however, are much more flexible than traditionally formed microbubbles as result of the nonionic triblock copolymer, which acts as a linker packed between lipids. This added flexibility of the nanobubbles reduces the resonant frequency or signal echogenicity to a point that make the nanobubbles detectable at frequencies as low as (1 MHz, e.g., 3.5 MHz) making the nanobubbles comparable to clinical agents but with the added benefit of small size.

Advantageously, the stabilized crosslinked nanobubble contrast agents had a substantially smaller diameter and a significant improvement in stability and retention of echogenic signal over 24 hours compared to similar stabilized nanobubbles that are not crosslinked. Moreover, in vivo analysis via ultrasound and fluorescence mediated tomography showed greater tumor extravasation and accumulation of the stabilized crosslinked nanobubble contrast agents compared to microbubbles.

Additionally, the lipid shell of the nanobubble also allows therapeutic agents and targeting moieties to be linked to, conjugated to, or encapsulated by the nanobubble. This permits the nanobubbles to be used as delivery vehicles in therapeutic applications as well as provides active targeting of the nanobubbles to the tissue or cells being treated.

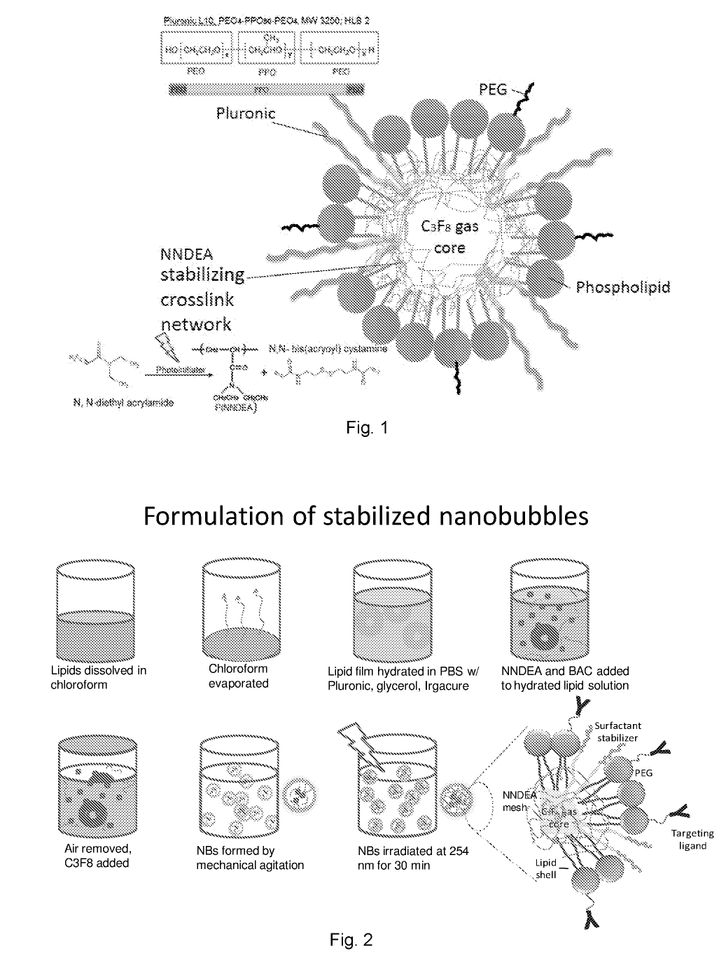

FIG. 1 illustrates a stabilized crosslinked nanobubble in accordance with one aspect of the application. The nanobubble can include a membrane or shell that defines an internal void. The internal void can include at least one gas. The membrane or shell can include at least one type of lipid, at least one type of nonionic triblock copolymer that is effective in controlling and/or reducing the size of the lipid nanobubbles without compromising nanobubble stability and in vitro and in vivo echogenicity, and an interpenetrating crosslinked biodegradable polymer.

“Nanobubble stability” can generally refer to the ability of the nanobubble to maintain its size in vitro and/or in vivo over time. For example, the nanobubble can maintain its size in vitro and/or in vivo over the course of minutes, days, weeks, or years. Additionally, nanobubble stability can refer to the polydispersity and/or zeta potential of the nanobubble. Polydispersity can refer to size distribution of the bubbles in solution, and zeta potential provides information on the stability of particle in suspension and is a function of particle surface charge. Nanobubbles described herein can have a polydispersity value of between about 0.1 and about 0.5, and a zeta potential of between about −30 mV and about −70 mV.

The nonionic triblock copolymers (e.g., poloxamers) can change the packing of the lipids in the nanobubble shell and allow the nanobubble size (diameter) to be tailored to as small as about 50 nm. It was found the nonionic triblock copolymers can reduce the nanobubble shell by increasing the lipid shell curvature and decreasing nanobubble surface tension without compromising bubble stability. At the same arc length the higher the bubble curvature, the smaller the bubble size. T nonionic triblock copolymer also enables a tighter packing but greater expansion (flexibility) of the lipid shell. Bubble resonance frequency is inversely proportional to bubble radius and related to shell elasticity, i.e., the more elastic the shell the lower the frequency. Therefore, the increase flexibility of the nanobubbles provided by the nonionic triblock copolymer leads to resonance at lower frequencies and highly echogenic bubbles.

In some embodiments, the nanobubble can have a size that facilitates extravasation of the nanobubble in cancer therapy or diagnosis. For example, the nanobubble can have a size (diameter) of about 30 nm to about 300 nm (e.g., about 50 nm to about 200 nm), depending upon the particular nonionic triblock copolymer and interpenetrating polymer as well as the method used to form the nanobubble (described in greater detail below).

In some embodiments, the nanobubble can include a hydrophilic outer domain that is at least partially defined by hydrophilic heads of the lipid and the nonionic triblock copolymer and a hydrophobic inner domain at least partially defined by hydrophobic tails of the lipid. The interpenetrating crosslinked biodegradable polymer can be non-covalently integrated into the hydrophobic domain of the nanobubble.

The at least one lipid comprising the membrane or shell can include any naturally-occurring, synthetic or semi-synthetic (i.e., modified natural) moiety that is generally amphipathic or amphiphilic (i.e., including a hydrophilic component and a hydrophobic component). Examples of lipids can include fatty acids, neutral fats, phospholipids, oils, glycolipids, surfactants, aliphatic alcohols, waxes, terpenes and steroids. Semi-synthetic or modified natural lipids can include natural lipids that have been chemically modified in some fashion. The at least one lipid can be neutrally-charged, negatively-charged (i.e., anionic), or positively-charged (i.e., cationic). Examples of anionic lipids can include phosphatidic acid, phosphatidyl glycerol, and fatty acid esters thereof, amides of phosphatidyl ethanolamine, such as anandamides and methanandamides, phosphatidyl serine, phosphatidyl inositol and fatty acid esters thereof, cardiolipin, phosphatidyl ethylene glycol, acidic lysolipids, sulfolipids and sulfatides, free fatty acids, both saturated and unsaturated, and negatively-charged derivatives thereof. Examples of cationic lipids can include N-[1-(2,3-dioleoyloxy)propyl]-N,N,N-trimethyl-ammonium chloride and common natural lipids derivatized to contain one or more basic functional groups.

Other examples of lipids, any one or combination of which may be used to form the membrane, can include: phosphocholines, such as 1-alkyl-2-acetoyl-sn-glycero 3-phosphocholines, and 1-alkyl-2-hydroxy-sn-glycero 3-phosphocholines; phosphatidylcholine with both saturated and unsaturated lipids, including dioleoylphosphatidylcholine, dimyristoylphosphatidylcholine, dipentadecanoylphosphatidylcholine, dilauroylphosphatidylcholine, dipalmitoylphosphatidylcholine (DPPC), dibehenoylglycerophosphocoline (DBPC), distearoylphosphatidylcholine (DSPC), and diarachidonylphosphatidylcholine (DAPC); phosphatidylethanolamines, such as dioleoylphosphatidylethanolamine, dipalmitoylphosphatidylethanolamine (DPPE), and distearoylphosphatidylethanolamine (DSPE); phosphatidylserine; phosphatidylglycerols, including distearoylphosphatidylglycerol (DSPG); phosphatidylinositol; sphingolipids, such as sphingomyelin; glycolipids, such as ganglioside GM1 and GM2; glucolipids; sulfatides; glycosphingolipids; phosphatidic acids, such as dipalmitoylphosphatidic acid (DPPA) and distearoylphosphatidic acid (DSPA); palmitic acid; stearic acid; arachidonic acid; oleic acid; lipids bearing polymers, such as chitin, hyaluronic acid, polyvinylpyrrolidone or polyethylene glycol (PEG); lipids bearing sulfonated mono-, di-, oligo- or polysaccharides; cholesterol, cholesterol sulfate, and cholesterol hemisuccinate; tocopherol hemisuccinate; lipids with ether and ester-linked fatty acids; polymerized lipids (a wide variety of which are well known in the art); diacetyl phosphate; dicetyl phosphate; stearylaamine; cardiolipin; phospholipids with short chain fatty acids of about 6 to about 8 carbons in length; phospholipids with medium chain fatty acids of about 10 to about 16 carbons in length; phospholipids with long chain fatty acids of about 18 to about 24 carbons in length; synthetic phospholipids with asymmetric acyl chains, such as, for example, one acyl chain of about 6 carbons and another acyl chain of about 12 carbons; ceramides; non-ionic liposomes including niosomes, such as polyoxyalkylene (e.g., polyoxyethylene) fatty acid esters, polyoxyalkylene (e.g., polyoxyethylene) fatty alcohols, polyoxyalkylene (e.g., polyoxyethylene) fatty alcohol ethers, polyoxyalkylene (e.g., polyoxyethylene) sorbitan fatty acid esters (such as, for example, the class of compounds referred to as TWEEN (commercially available from ICI Americas, Inc., Wilmington, Del.), glycerol polyethylene glycol oxystearate, glycerol polyethylene glycol ricinoleate, alkyloxylated (e.g., ethoxylated) soybean sterols, alkyloxylated (e.g., ethoxylated) castor oil, polyoxyethylene-polyoxypropylene polymers, and polyoxyalkylene (e.g., polyoxyethylene) fatty acid stearates; sterol aliphatic acid esters including cholesterol sulfate, cholesterol butyrate, cholesterol isobutyrate, cholesterol palmitate, cholesterol stearate, lanosterol acetate, ergosterol palmitate, and phytosterol n-butyrate; sterol esters of sugar acids including cholesterol glucuronide, lanosterol glucuronide, 7-dehydrocholesterol glucuronide, ergosterol glucuronide, cholesterol gluconate, lanosterol gluconate, and ergosterol gluconate; esters of sugar acids and alcohols including lauryl glucuronide, stearoyl glucuronide, myristoyl glucuronide, lauryl gluconate, myristoyl gluconate, and stearoyl gluconate; esters of sugars and aliphatic acids including sucrose laurate, fructose laurate, sucrose palmitate, sucrose stearate, glucuronic acid, gluconic acid and polyuronic acid; saponins including sarsasapogenin, smilagenin, hederagenin, oleanolic acid, and digitoxigenin; glycerol dilaurate, glycerol trilaurate, glycerol dipalmitate, glycerol and glycerol esters including glycerol tripalmitate, glycerol distearate, glycerol tristearate, glycerol dimyristate, glycerol trimyristate; long chain alcohols including n-decyl alcohol, lauryl alcohol, myristyl alcohol, cetyl alcohol, and n-octadecyl alcohol; 6-(5-cholesten-3β-yloxy)-1-thio-β-D-galactopyranoside; digalactosyldiglyceride; 6-(5-cholesten-3β-yloxy)hexyl-6-amino-6-deoxy-1-thio-β-D-galactopyranoside; 6-(5-cholesten-3β-yloxy)hexyl-6-amino-6-deoxyl-1-thio-α-D-mannopyranoside; 12-(((7′-diethylaminocoumarin-3-yl)carbonyl)methylamino)octadecanoic acid; N-[12-(((7′-diethylaminocoumarin-3-yl)carbonyl)methylamino)octadecanoyl]-2-aminopalmitic acid; cholesteryl(4′-trimethylammonio)butanoate; N-succinyldioleoylphosphatidylethanolamine; 1,2-dioleoyl-sn-glycerol; 1,2-dipalmitoyl-sn-3-succinylglycerol; 1,3-dipalmitoyl-2-succinylglycerol; 1-hexadecyl-2-palmitoylglycerophosphoethanolamine and palmitoylhomocysteine; and/or any combinations thereof.

In some embodiments, the lipids can include a mixture of phospholipids having varying acyl chain lengths. For example, the lipids can include a mixture of at least two of DPPC, DBPC, DPPE, or DPPA. In one embodiment, the lipids can include a mixture of DPPC, DPPE, and DPPA at a ratio of, for example, about 4:1.4:1. In another embodiment, the lipids can include a mixture of DBPC, DPPE, and DPPA at a ratio of, for example, about 6:2:1. Advantageously, increasing the length of the acyl chain of the most predominant lipid in the mixture from 16 to 22 carbons (i.e., from DPPC to DBPC), while maintaining about the same molar ratios of all lipids in the formulation resulted in a fourfold improvement in half-life of the nanobubbles.

In some embodiments, the at least one nonionic triblock copolymer used to form the membrane can include an amphiphilic surfactant, such as a poloxamer, poloxamine, meroxapol, and/or combination thereof. In one example, the at least one nonionic triblock copolymer can comprise a poloxamer. The poloxamer can include any one or combination of a series of block copolymers of ethylene oxide and propylene oxide. The poly(oxyethylene) (PEO) and poly(oxypropylene) (PPO) segments may be hydrophilic and hydrophobic, respectively. The poloxamer may be a liquid, a paste, or a solid, and may have a molecular weight that ranges, for example, from about 1000 Daltons to about 3500 Daltons, although poloxamers having molecular weights greater or less than the these molecular weights can potentially be used. The concentration of nonionic triblock copolymer in the lipid nanobubble can be about 0.06 mg/ml to about 1 mg/ml.

The basic chemical formula of the poloxamer may be HO—(C₂H₄O)ₐ(C₃H₆O)_{b}(C₂H₄O)ₐ—H, where “a” and “b” represent repeating units of PEO and PPO, respectively. In some embodiments, “a” can be from 2 to 130 and “b” can be from 15 to 67. In one example, the poloxamer may have the chemical formula of HO—(C₂H₄O)₂(C₃H₆O)₃₁(C₂H₄O)₂—H. In another example of the present invention, the poloxamer may have the chemical formula of HO—(C₂H₄O)₃(C₃H₆O)₄₃(C₂H₄O)₃—H.

The poloxamer may be commercially available under various trade names including, for example, LUTROL, PLURONIC, SYNPERONIC (ICI), EMKALYX, PLURACARE, and PLURODAC. Examples of the PLURONIC series can include, but are not limited to, PLURONIC L10 (avg. M_{w}: 3200), PLURONIC L81 (avg. M_{w}: 2750), PLURONIC L61 (avg. M_{w}: 2000), PLURONIC L72 (avg. M_{w}: 2750), PLURONIC L62 (avg. M_{w}: 2500), PLURONIC L42 (avg. M_{w}: 1630), PLURONIC L63 (avg. M_{w}: 2650), PLURONIC L43 (avg. M_{w}: 1850), PLURONIC L64 (avg. M_{w}: 2900), PLURONIC L44 (avg. M_{w}: 2200), and PLURONIC L35 (avg. M_{w}: 1900). Other commercially available poloxamers can include compounds that are block copolymers of polyethylene and polypropylene glycol, such as SYNPERONIC L121, SYNPERONIC L122, SYNPERONIC P104, SYNPERONIC P105, SYNPERONIC P123, SYNPERONIC P85, SYNPERONIC P94, and compounds that are nonylphenyl polyethylene glycol, such as SYNPERONIC NP10, SYNPERONIC NP30 and SYNPERONIC NP5.

In another embodiment, the at least one nonionic triblock copolymer can comprise a poloxamine. The poloxamine can include a polyalkoxylated symmetrical block copolymer prepared from an ethylene diamine initiator. Poloxamines are synthesized using the same sequential order of addition of alkylene oxides as used to synthesize poloxamers. Structurally, the poloxamines can include four alkylene oxide chains and two tertiary nitrogen atoms, at least one of which is capable of forming a quaternary salt. Poloxamines can also be terminated by primary hydroxyl groups. Examples of poloxamines can include, but are not limited to, the TETRONIC and/or TETRONIC R series produced by BASF. For example, poloxamines can include TETRONIC 904, TETRONIC 908, TETRONIC 1107, TETRONIC 90R4, TETRONIC 1304, TETRONIC 1307 and TETRONIC T1501.

In another aspect of the application, the at least one nonionic triblock copolymer can include a meroxapol. Meroxapols can include a symmetrical block copolymer consisting of a core of PEG polyoxypropylated to both its terminal hydroxyl groups, i.e., conforming to the general type (PPG)ₓ-(PEG)_{y}-(PPG)ₓ, and being formed by an ethylene glycol initiator. Examples of meroxapols can include, but are not limited to, MEROXAPOL 105, MEROXAPOL 108, MEROXAPOL 172, MEROXAPOL 174, MEROXAPOL 252, MEROXAPOL 254, MEROXAPOL 258 and MEROXAPOL 311.

The interpenetrating cross-linking biodegradable polymer can formed from a polymerizable acrylamide monomer, such as N, N-diethyl acrylamide (NNDEA), that is reacted with or cross-linked with a bifunctional crosslinker, such as N, N-bis(acryoyl)cystamine (BAC), in the presence of an initiator, such as a radical photoinitiator (e.g., IRGACURE 2959). Incorporation of cross-linking agents into the nanobubble membrane was found to increase the stability of pluronic polymeric micelles below their critical micelle concentration (CMC). In these nanobubbles, the hydrophobic network is non-covalently integrated into the inner ring or the hydrophobic domain of the nanobubble to improve structural stability while retaining membrane flexibility and reduce diffusion of hydrophobic gas from the core.

In some embodiments, the acrylamide monomer can include at least one of N-(n-octadecyl)acrylamide, acrylamide, N-benzylmethacrylamide, N,N-diethylacrylamide, N,N-dimethylacrylamide, N,N-dimethylmethacrylamide, N,N-diphenyl methacrylamide, N(n-dodecyl)methacrylamide, N-(tert-octyl)acrylamide, N-iso-propylacrylamide, N-[2-N,N-Dimethylamino)-ethyl]methacrylamide, N-[3-(N,N-Dimethylamino)-propyl] acrylamide, or N-[3-(N,N-Dimethylamino)-propyl] methacrylamide, N-tert-butylacrylamide, N-(butoxymethyl)acrylamide, diacetoneacrylamide, dodecylacrylamide, ethylenebisacrylamide, n-(hydroxymethyl)acrylamide, methylenebisacrylamide, phenyl acrylamide, or combinations thereof. The particular acrylamide monomer selected or combination of acrylamide monomers can affect the stability of the nanobubbles. More hydrophic acrylamide monomers can form more hydrophic acylamide polymers that can enhance stability of the nanobubbles compared to less hydrophobic acrylamide monomers and polymers.

The membrane defining the nanobubble can be concentric or otherwise and have a unilamellar configuration (i.e., comprised of one monolayer or bilayer), an oligolamellar configuration (i.e., comprised of about two or about three monolayers or bilayers), or a multilamellar configuration (i.e., comprised of more than about three monolayers or bilayers). The membrane can be substantially solid (uniform), porous, or semi-porous.

The internal void defined by the membrane can include at least one gas. The gas can have a low solubility in water and be, for example, a perfluorocarbon, such as perfluoropropane (e.g., octafluoropropane). The internal void can also include other gases, such as carbon dioxide, air, nitrogen, and helium.

In some embodiments, the nanobubbles can include a linker to link the targeting moiety and/or bioactive agent to the membrane of the nanobubble. The linker can be of any suitable length and contain any suitable number of atoms and/or subunits. The linker can include one or combination of chemical and/or biological moieties. Examples of chemical moieties can include alkyl groups, methylene carbon chains, ether, polyether, alkyl amide linkers, alkenyl chains, alkynyl chains, disulfide groups, and polymers, such as poly(ethylene glycol) (PEG), functionalized PEG, PEG-chelant polymers, dendritic polymers, and combinations thereof. Examples of biological moieties can include peptides, modified peptides, streptavidin-biotin or avidin-biotin, polyaminoacids (e.g., polylysine), polysaccharides, glycosaminoglycans, oligonucleotides, phospholipid derivatives, and combinations thereof.

The stabilized nanobubbles can also include other materials, such as liquids, oils, bioactive agents, diagnostic agents, and/or therapeutic agents. The materials can be encapsulated by the membrane and/or linked or conjugated to the membrane.

Bioactive agents encapsulated by and/or linked to the membrane can include any substance capable of exerting a biological effect in vitro and/or in vivo. Examples of bioactive agents can include, but are not limited to, chemotherapeutic agents, biologically active ligands, small molecules, DNA fragments, DNA plasmids, interfering RNA molecules, such as siRNAs, oligonucleotides, and DNA encoding for shRNA. Diagnostic agents can include any substance that may be used for imaging a region of interest (ROI) in a subject and/or diagnosing the presence or absence of a disease or diseased tissue in a subject. Therapeutic agents can refer to any therapeutic or prophylactic agent used in the treatment (including the prevention, diagnosis, alleviation, or cure) of a malady, affliction, condition, disease or injury in a subject. It will be appreciated that the membrane can additionally or optionally include proteins, carbohydrates, polymers, surfactants, and/or other membrane stabilizing materials, any one or combination of which may be natural, synthetic, or semi-synthetic.

In some embodiments, the bioactive agent can include a therapeutic agent, such as a chemotherapeutic agent, an anti-proliferative agent, an anti-microbial agent, a biocidal agent, and/or a biostatic agent. The therapeutic agent can be encapsulated by and/or linked to the membrane of the nanobubble.

In some embodiments, the membrane can additionally or optionally include at least one targeting moiety that is capable of targeting and/or adhering the nanobubble to a cell or tissue of interest. In some embodiments, the targeting moiety can comprise any molecule, or complex of molecules, which is/are capable of interacting with an intracellular, cell surface, or extracellular biomarker of the cell. The biomarker can include, for example, a cellular protease, a kinase, a protein, a cell surface receptor, a lipid, and/or fatty acid. Other examples of biomarkers that the targeting moiety can interact with include molecules associated with a particular disease. For example, the biomarkers can include cell surface receptors implicated in cancer development, such as CA-125 receptor, epidermal growth factor receptor, and transferrin receptor. The targeting moiety can interact with the biomarkers through non-covalent binding, covalent binding, hydrogen binding, van der Waals forces, ionic bonds, hydrophobic interactions, electrostatic interaction, and/or combinations thereof.

The targeting moiety can include, but is not limited to, synthetic compounds, natural compounds or products, macromolecular entities, bioengineered molecules (e.g., polypeptides, lipids, polynucleotides, antibodies, antibody fragments), and small entities (e.g., small molecules, neurotransmitters, substrates, ligands, hormones and elemental compounds).

In one example, the targeting moiety can comprise an antibody, such as a monoclonal antibody, a polyclonal antibody, or a humanized antibody, including without limitation: Fv fragments, single chain Fv (scFv) fragments, Fab′ fragments, F(ab′)2 fragments, single domain antibodies, camelized antibodies and antibody fragments, humanized antibodies and antibody fragments, and multivalent versions of the foregoing; multivalent targeting moieties including without limitation: monospecific or bispecific antibodies, such as disulfide stabilized Fv fragments, scFv tandems ((scFv)₂ fragments), diabodies, tribodies or tetrabodies, which typically are covalently linked or otherwise stabilized (i.e., leucine zipper or helix stabilized) scFv fragments; and receptor molecules, which naturally interact with a desired target molecule.

Preparation of antibodies may be accomplished by any number of well-known methods for generating antibodies. These methods typically include the step of immunization of animals, typically mice, with a desired immunogen (e.g., a desired target molecule or fragment thereof). Once the mice have been immunized, and boosted one or more times with the desired immunogen(s), antibody-producing hybridomas may be prepared and screened according to well known methods. See, for example, Kuby, Janis, Immunology, Third Edition, pp. 131-139, W.H. Freeman & Co. (1997), for a general overview of monoclonal antibody production, that portion of which is incorporated herein by reference.

The targeting moiety need not originate from a biological source. The targeting moiety may, for example, be screened from a combinatorial library of synthetic peptides. One such method is described in U.S. Pat. No. 5,948,635, incorporated herein by reference, which describes the production of phagemid libraries having random amino acid insertions in the pIII gene of M13. This phage may be clonally amplified by affinity selection.

The immunogens used to prepare targeting moieties having a desired specificity will generally be the target molecule, or a fragment or derivative thereof. Such immunogens may be isolated from a source where they are naturally occurring or may be synthesized using methods known in the art. For example, peptide chains may be synthesized by 1-ethyl-3-[dimethylaminoproply]carbodiimide (EDC)-catalyzed condensation of amine and carboxyl groups. In certain embodiments, the immunogen may be linked to a carrier bead or protein. For example, the carrier may be a functionalized bead such as SASRIN resin commercially available from Bachem, King of Prussia, Pa. or a protein such as keyhole limpet hemocyanin (KLH) or bovine serum albumin (BSA). The immunogen may be attached directly to the carrier or may be associated with the carrier via a linker, such as a non-immunogenic synthetic linker (for example, a polyethylene glycol (PEG) residue, amino caproic acid or derivatives thereof) or a random, or semi-random polypeptide.

In certain embodiments, it may be desirable to mutate the binding region of the polypeptide targeting moiety and select for a targeting moiety with superior binding characteristics as compared to the un-mutated targeting moiety. This may be accomplished by any standard mutagenesis technique, such as by PCR with Taq polymerase under conditions that cause errors. In such a case, the PCR primers could be used to amplify scFv-encoding sequences of phagemid plasmids under conditions that would cause mutations. The PCR product may then be cloned into a phagemid vector and screened for the desired specificity, as described above.

In other embodiments, the targeting moiety may be modified to make them more resistant to cleavage by proteases. For example, the stability of targeting moiety comprising a polypeptide may be increased by substituting one or more of the naturally occurring amino acids in the (L) configuration with D-amino acids. In various embodiments, at least 1%, 5%, 10%, 20%, 50%, 80%, 90% or 100% of the amino acid residues of targeting moiety may be of the D configuration. The switch from L to D amino acids neutralizes the digestion capabilities of many of the ubiquitous peptidases found in the digestive tract. Alternatively, enhanced stability of a targeting moiety comprising a peptide bond may be achieved by the introduction of modifications of the traditional peptide linkages. For example, the introduction of a cyclic ring within the polypeptide backbone may confer enhanced stability in order to circumvent the effect of many proteolytic enzymes known to digest polypeptides in the stomach or other digestive organs and in serum. In still other embodiments, enhanced stability of a targeting moiety may be achieved by intercalating one or more dextrorotatory amino acids (such as, dextrorotatory phenylalanine or dextrorotatory tryptophan) between the amino acids of targeting moiety. In exemplary embodiments, such modifications increase the protease resistance of a targeting moiety without affecting the activity or specificity of the interaction with a desired target molecule.

In certain embodiments, the antibodies or variants thereof may be modified to make them less immunogenic when administered to a subject. For example, if the subject is human, the antibody may be “humanized”; where the complimentarily determining region(s) of the hybridoma-derived antibody has been transplanted into a human monoclonal antibody, for example as described in Jones, P. et al. (1986), Nature, 321, 522-525 or Tempest et al. (1991), Biotechnology, 9, 266-273. Also, transgenic mice, or other mammals, may be used to express humanized antibodies. Such humanization may be partial or complete.

In certain embodiments, a targeting moiety as described herein may comprise a homing peptide, which selectively directs the nanobubble to a targeted cell. Homing peptides for a targeted cell can be identified using various methods well known in the art. Many laboratories have identified the homing peptides that are selective for cells of the vasculature of brain, kidney, lung, skin, pancreas, intestine, uterus, adrenal gland, retina, muscle, prostate, or tumors. See, for example, Samoylova et al., 1999, Muscle Nerve, 22:460; Pasqualini et al., 1996 Nature, 380:364; Koivunen et al., 1995, Biotechnology, 13:265; Pasqualini et al., 1995, J. Cell Biol., 130:1189; Pasqualini et al., 1996, Mole. Psych., 1:421, 423; Rajotte et al., 1998, J. Clin. Invest., 102:430; Rajotte et al., 1999, J. Biol. Chem., 274:11593. See, also, U.S. Pat. Nos. 5,622,6999; 6,068,829; 6,174,687; 6,180,084; 6,232,287; 6,296,832; 6,303,573; and 6,306,365.

Phage display technology provides a means for expressing a diverse population of random or selectively randomized peptides. Various methods of phage display and methods for producing diverse populations of peptides are well known in the art. For example, methods for preparing diverse populations of binding domains on the surface of a phage have been described in U.S. Pat. No. 5,223,409. In particular, phage vectors useful for producing a phage display library as well as methods for selecting potential binding domains and producing randomly or selectively mutated binding domains are also provided in U.S. Pat. No. 5,223,409. Similarly, methods of producing phage peptide display libraries, including vectors and methods of diversifying the population of peptides that are expressed, are also described in Smith et al., 1993, Meth. Enzymol., 217:228-257, Scott et al., Science, 249:386-390, and two PCT publications WO 91/07141 and WO 91/07149. Phage display technology can be particularly powerful when used, for example, with a codon based mutagenesis method, which can be used to produce random peptides or randomly or desirably biased peptides (see, e.g., U.S. Pat. No. 5,264,563). These or other well-known methods can be used to produce a phage display library, which can be subjected to the in vivo phage display method in order to identify a peptide that homes to one or a few selected tissues.

In vitro screening of phage libraries has previously been used to identify peptides that bind to antibodies or cell surface receptors (see, e.g., Smith, et al., 1993, Meth. Enzymol., 217:228-257). For example, in vitro screening of phage peptide display libraries has been used to identify novel peptides that specifically bind to integrin adhesion receptors (see, e.g., Koivunen et al., 1994, J. Cell Biol. 124:373-380), and to the human urokinase receptor (Goodson, et al., 1994, Proc. Natl. Acad. Sci., USA 91:7129-7133).

In certain embodiments, the targeting moiety may comprise a receptor molecule, including, for example, receptors, which naturally recognize a specific desired molecule of a target cell. Such receptor molecules include receptors that have been modified to increase their specificity of interaction with a target molecule, receptors that have been modified to interact with a desired target molecule not naturally recognized by the receptor, and fragments of such receptors (see, e.g., Skerra, 2000, J. Molecular Recognition, 13:167-187). A preferred receptor is a chemokine receptor. Exemplary chemokine receptors have been described in, for example, Lapidot et al, 2002, Exp Hematol, 30:973-81 and Onuffer et al, 2002, Trends Pharmacol Sci, 23:459-67.

In other embodiments, the targeting moiety may comprise a ligand molecule, including, for example, ligands which naturally recognize a specific desired receptor of a target cell. Such ligand molecules include ligands that have been modified to increase their specificity of interaction with a target receptor, ligands that have been modified to interact with a desired receptor not naturally recognized by the ligand, and fragments of such ligands.

In still other embodiments, the targeting moiety may comprise an aptamer. Aptamers are oligonucleotides that are selected to bind specifically to a desired molecular structure of the target cell. Aptamers typically are the products of an affinity selection process similar to the affinity selection of phage display (also known as in vitro molecular evolution). The process involves performing several tandem iterations of affinity separation, e.g., using a solid support to which the diseased immunogen is bound, followed by polymerase chain reaction (PCR) to amplify nucleic acids that bound to the immunogens. Each round of affinity separation thus enriches the nucleic acid population for molecules that successfully bind the desired immunogen. In this manner, a random pool of nucleic acids may be “educated” to yield aptamers that specifically bind target molecules. Aptamers typically are RNA, but may be DNA or analogs or derivatives thereof, such as, without limitation, peptide nucleic acids (PNAs) and phosphorothioate nucleic acids.

In yet other embodiments, the targeting moiety may be a peptidomimetic. By employing, for example, scanning mutagenesis to map the amino acid residues of a protein, which is involved in binding other proteins, peptidomimetic compounds can be generated that mimic those residues, which facilitate the interaction. Such mimetics may then be used as a targeting moiety to deliver the nanobubble to a target cell. For instance, non-hydrolyzable peptide analogs of such resides can be generated using benzodiazepine (e.g., see Freidinger et al. in Peptides: Chemisty and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988), keto-methylene pseudopeptides (Ewenson et al., 1986, J Med Chem 29:295; and Ewenson et al., in Peptides: Structure and Function (Proceedings of the 9th American Peptide Symposium) Pierce Chemical Co. Rockland, Ill., 1985), b-turn dipeptide cores (Nagai et al., 1985, Tetrahedron Lett 26:647; and Sato et al., 1986, J Chem Soc Perkin Trans 1:1231), and β-aminoalcohols (Gordon et al., 1985, Biochem Biophys Res Cummun 126:419; and Dann et al., 1986, Biochem Biophys Res Commun 134:71).

By way of example, where the cell targeted comprises an ovarian cancer cell, the targeting moiety can comprise an antibody or peptide to human CA-125R. Over expression of CA-125 has implication in ovarian cancer cells. Alternatively, where the cell targeted comprises a malignant cancer, such as glioblastoma, the targeting moiety can comprise an antibody or peptide to extracellular growth factor receptor (EGFR) and/or human transferrin receptor (TfR). Overexpression of EGFR and TfR has been implicated in the malignant phenotype of tumor cells. The overexpression of these receptors also leads to activation of other genes that promote cancer growth through such means as invasion and metastasis, as well as resistance to chemotherapy and radiotherapy. The imaging of cancer cells expressing EGFR and TfR can provide a molecular signature of the malignancy or progression of such cells.

FIG. 2 is a schematic diagram illustrating a method of forming a composition comprising at least one stabilized nanobubble. Although previous methodologies have been developed to reduce bubble size, most of these strategies involve manipulations of microbubbles post formation, such as gradient separation by gravitational forces or by physical filtration or floatation. While effective for selecting nanosized bubbles, these methods introduce potential for sample contamination, reduce bubble yield and stability, and waste stock materials in addition to being labor intensive. Unlike the post formation methods of the prior art, the method described herein can be used to form nanobubbles without any manipulation of the bubbles post formation and thereby avoid or mitigate the potential for sample contamination, reduced bubble yield and stability, waste of stock materials, and labor intensive.

In the first step of the method, at least one lipid can be dissolved in a solvent to produce a lipid-solvent solution. The lipid(s) dissolved in the solvent can include any one or combination of those described above. It will be appreciated that other materials can be dissolved in the solvent to stabilize the membrane, such as proteins, carbohydrates, polymers, surfactants, and/or other membrane stabilizing materials. The solvent can include any organic solvent, such as chloroform, methylene chloride, ethylene chloride, ethylene dichloride, ethyl acetate, methylchloroform, tetrahydrofuran or benzene. In one example, the lipids DPPC (1,2-Dipalmitoyl-sn-Glycero-3-Phosphocholine), DPPA (1,2 Dipalmitoyl-sn-Glycero-3-Phosphate), DPPE (1,2-dipalmitoyl-sn-glycero-3-phosphoethanolamine) (Avanti Polar Lipids, Pelham, Ala.), and mPEG-DSPE (1,2-Distearoyl-phosphatidylethanol amine-methyl-poly ethylene glycol conjugate-2000) (Laysan Lipids, Arab, Ala.) were dissolved in chloroform in a 6.1:2:1:1 mass ratio. In another example, the lipids DBPC (1,2-Dibehenoyl-sn-Glycero-3-Phosphocholine), DPPA (1,2 Dipalmitoyl-sn-Glycero-3-Phosphate), DPPE (1,2-dipalmitoyl-sn-glycero-3-phosphoethanolamine) (Avanti Polar Lipids, Pelham, Ala.), and mPEG-DSPE (1,2-Distearoyl-phosphatidylethanol amine-methyl-poly ethylene glycol conjugate-2000) (Laysan Lipids, Arab, Ala.).

After producing the lipid-solvent solution, the solvent can be evaporated at a temperature and for a time sufficient to form a film. For example, the lipid-solvent solution can be placed in an open container (e.g., an evaporating dish, beaker, vial, etc.) and then set on a heat source (e.g., incubator, steam bath, hot plate, heating mantle, sand bath, etc.) at a temperature and for a time sufficient to evaporate the solvent and form the film.

The resultant film can be hydrated to form at least one lipid vesicle. The term “vesicle” can refer to any entity that is generally characterized by the presence of one or more walls or membranes that define one or more internal voids. The film can be hydrated by contacting an amount of at least one buffer solution (e.g., about 1×PBS) with an organic compound (e.g., glycerol). The hydration buffer/organic compound solution can then be placed in a container (e.g., a vial) and stirred or shaken at a temperature and for a time sufficient to produce at least one lipid vesicle. In one example, a lipid-solvent solution comprising DPPC/DPPA/DPPE dissolved in chloroform can be contacted with a hydration PBS/glycerol solution, placed in a vial, and then placed in an incubator-shaker at about 37° C. and at about 120 rpm for about 60 minutes.

The hydration buffer/organic compound solution can also include at least one nonionic triblock polymer (e.g., 0.6 mg/ml Pluronic L10 solution) and polymer initiator 0.5% Irgacure 2959. The at least one nonionic triblock polymer can be added at a concentration and/or at a lipid:nonionic triblock polymer ratio effective to control the size of the nanobubble and impart the nanobubble with in vitro and in vivo echogenicity. In one example, PLURONIC L10 can be added to the hydration buffer/organic compound solution at a concentration of between about 0.1 mg/mL and about 1 mg/mL and at a lipid:PLURONIC L10 ratio of about 2:1 to about 50:1.

Following the addition of the at least one nonionic triblock copolymer and initiator, an acrylamide monomer (e.g., NNDEA) and crosslinker (BAC) can be added to the hydrated lipid solution. The air can removed from a sealed vial containing the hydrated lipid solution and replaced with octafluoropropane until the vial pressure equalized. The resultant solution can then be shaken or stirred for a time (e.g., about 45 seconds) sufficient to form the nanobubble. Finally, the nanobubbles can be irradiated at wavelength effective to promote activation of the polymer initiator and polymerization and crosslinking of the acrylamide monomers.

The stabilized nanobubble composition so formed can be administered to a subject for diagnostic, therapeutic, and/or theranostic applications. In some embodiments, the nanobubbles can be administered to a subject for imaging at least one region of interest (ROI) of the subject. The ROI can include a particular area or portion of the subject and, in some instances, two or more areas or portions throughout the entire subject. The ROI can include, for example, pulmonary regions, gastrointestinal regions, cardiovascular regions (including myocardial tissue), renal regions, as well as other bodily regions, tissues, lymphocytes, receptors, organs and the like, including the vasculature and circulatory system, and as well as diseased tissue, including neoplastic or cancerous tissue. The ROI can include regions to be imaged for both diagnostic and therapeutic purposes. The ROI is typically internal; however, it will be appreciated that the ROI may additionally or alternatively be external.

In some embodiments, the nanobubbles used to image the ROI can be formulated such that the internal void of at least one of the nanobubbles includes at least one contrast agent. For example, a contrast agent (in either liquid or gaseous form) can be contacted with the hydrated lipid/nonionic triblock copolymer solution under conditions effective to entrap the contrast agent in the internal void of the nanobubble. For instance, sealed vials containing a lipid 1,2-dipalmitoyl-sn-glycero-3-phosphocholine/dipalmitoyl-sn-glycero-3-phosphoethanolamine/dipalmitoyl-sn-glycero-3-phosphate (DPPC/DPPA/DPPE)/poloxamer solution/acrylamide monomer/crosslinking agent can have the air withdrawn by a syringe and then octafluoropropane added until the pressure in the vial is equalized. Other examples of contrast agents (besides octafluoropropane) that may be incorporated into the nanobubbles are known in the art and can include stable free radicals, such as, stable nitroxides, as well as compounds comprising transition, lanthanide and actinide elements, which may, if desired, be in the form of a salt or may be covalently or non-covalently bound to complexing agents, including lipophilic derivatives thereof, or to proteinaceous macromolecules.

Since nanobubble size may influence biodistribution, the size of the nanobubbles can be selected depending upon the region of interest (ROI) of the subject. For a ROI comprising an organ (e.g., a liver or kidney) the size of the nanobubbles may be greater than for a ROI comprising tumor tissue. Where the ROI comprises, for example, tumor tissue and differentiation between the tumor tissue and normal or healthy tissue is sought, smaller nanobubbles may be needed to penetrate the smaller venuoles and capillaries comprising the tumor tissue. It should be appreciated that the nanobubbles can comprise additional constituents, such as targeting ligands to facilitate homing of the nanobubbles to the ROI.

The nanobubble composition can be administered to the subject via any known route, such as via an intravenous injection. By way of example, a composition comprising a plurality of octafluoropropane-containing nanobubbles can be intravenously administered to a subject that is known to or suspected of having a tumor.

At least one image of the ROI can be generated using an imaging modality. The imaging modality can include one or combination of known imaging techniques capable of visualizing the nanobubbles. Examples of imaging modalities can include ultrasound (US), magnetic resonance imaging (MRI), nuclear magnetic resonance (NMR), computed topography (CT), electron spin resonance (ESR), nuclear medical imaging, optical imaging, and positron emission topography (PET). The imaging modality can then be operated to generate a visible image of the ROI. In a subject known to or suspected of having a tumor, for example, an ultrasonic transducer can be applied to at least a portion of the ROI to image the target tissue. A visible image of the tumor can then be obtained, such that the presence, absence, and/or extent of a particular neoplastic disorder can be ascertained. It will be appreciated that the imaging modality may be used to generate a baseline image prior to administration of the composition. In this case, the baseline and post-administration images can be compared to ascertain the presence, absence, and/or extent of a particular disease or condition.

In other embodiments, the nanobubbles can be administered to a subject to treat and/or image a neoplastic disease in subject. Neoplastic diseases treatable by the present invention can include disease states in which there are cells and/or tissues which proliferate abnormally. One example of a neoplastic disease is a tumor. The tumor can include a solid tumor, such as a solid carcinoma, sarcoma or lymphoma, and/or an aggregate of neoplastic cells. The tumor may be malignant or benign, and can include both cancerous and pre-cancerous cells.

A composition comprising the stabilized nanobubbles can be formulated for administration (e.g., injection) to a subject diagnosed with at least one neoplastic disorder. The nanobubbles can be formulated according to method as described above and include, for example, at least one therapeutic agent or bioactive agent as well as targeting moiety to target the neoplastic cells.

By way of example, the stabilized nanobubbles can be targeted to ovarian cancer cells by conjugating a ligand that this is specific for the CA-125 receptor that is over expressed on ovarian cancer cells. The targeted stabilized nanobubbles can be formulated with at least one lipid that is conjugated to biotin. The nanobubbles can then be combined with streptavidin and a biotinylated anti-CA-125 antibody (e.g., MUC16, ab90346), which will then become conjugated to biotin of the lipid.

The location(s) where the nanobubble composition is administered to the subject may be determined based on the subject's individual need, such as the location of the neoplastic cells (e.g., the position of a tumor, the size of a tumor, and the location of a tumor on or near a particular organ). For example, the composition may be injected intravenously into the subject. It will be appreciated that other routes of injection may be used including, for example, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal routes.

Since nanobubble size may influence biodistribution, the size of the nanobubbles can be selected depending on the neoplastic disorder being imaged and/or treated. Where the neoplastic disorder comprises tumor tissue, smaller nanobubbles may be needed to penetrate the smaller venuoles and capillaries comprising the tumor tissue.

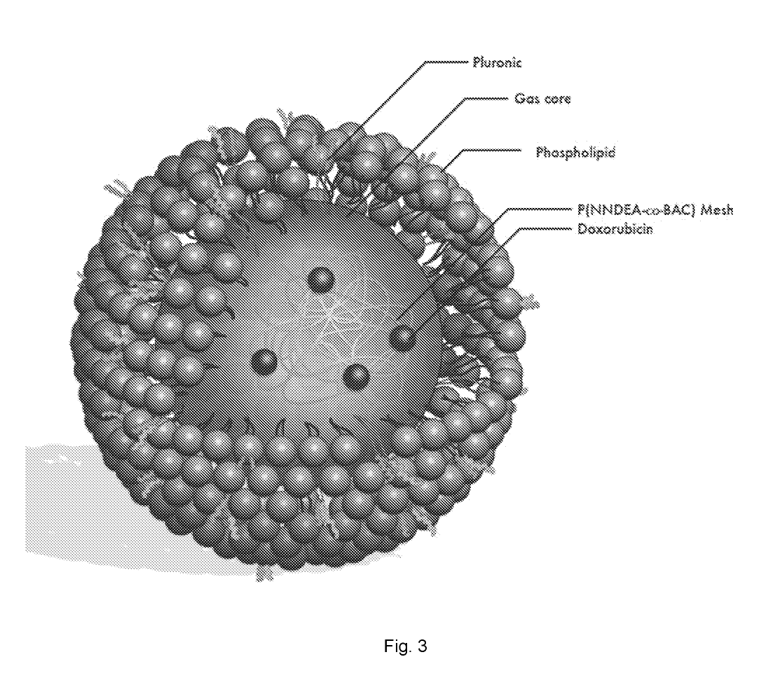

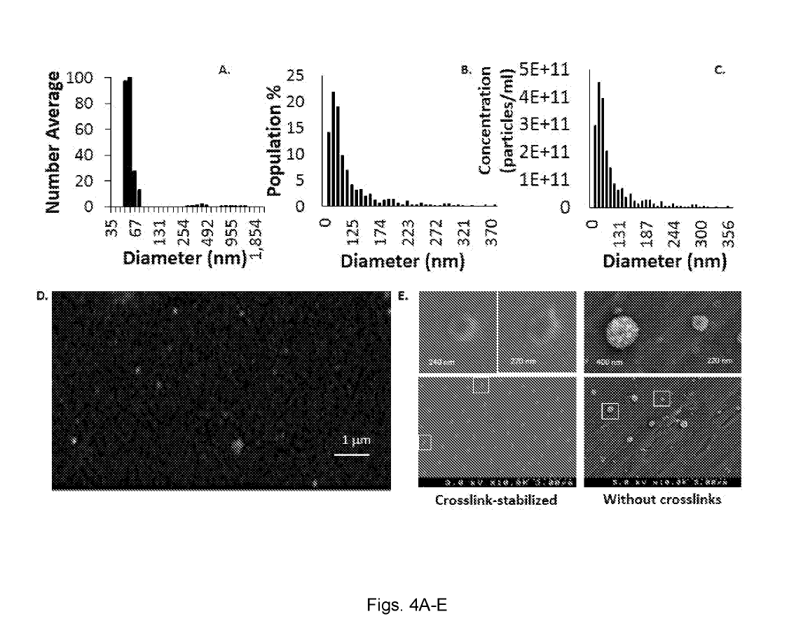

In some embodiments as shown schematically in FIG. 3, a bioactive agent and/or therapeutic agent, such as a chemotherapeutic (e.g., doxorubicin) can be loaded into the nanobubble during nanobubble formation to provide a stabilized crosslinked drug loaded nanobubble. The stabilized crosslinked drug loaded nanobubble can be responsive to energy, from a remote source that is effective to release the therapeutic agent from the nanobubble after administering the nanobubble to a subject. The remote source can be external or remote from a subject, which allows non-invasive remote release of the therapeutic agent to the subject. Advantageously, nanobubbles that allow remote release of the therapeutic agent, such as a chemotherapeutic agent (e.g., doxorubicin) can target or be targeted to specific cells or tissue of subject, such as tumors, cancers, and metastases, by systemic administration (e.g., intravenous, intravascular, or intraarterial infusion) to the subject and once targeted to the cells or tissue remotely released to specifically treat the targeted cells or tissue of subject (e.g., tumors, cancers, and metastasis). Targeting and selective release of the chemotherapeutic agents to malignant cancer metastases allows treatment of such metastases using chemotherapeutics, which would provide an otherwise neglible effect if not targeted and remotely released using the nanobubbles described herein.

In some embodiments, ultrasound can be used as a remote source to provide locoregional destruction or fragmentation of the nanobubbles to release the therapeutic agent (e.g., chemotherapeutic agent) provided in the nanobubbles to the tissue or region of interest (e.g., cancer or tumor). Advantageously, ultrasound can release the chemotherapeutic agent from the nanobubbles and enhance the anti-tumor efficacy chemotherapeutic agent by ultrasonic cavitation effects, sound effect and other effects. The radiation ultrasound also improves the cell membrane permeability of the chemotherapeutic resulting in much more chemotherapeutic agent in the tumor cells.

It will be appreciated that other remote sources can be used to provide locoregional destruction or fragmentation of the nanobubbles to release the therapeutic agent. These other remote sources can include, for example, high intensity focused ultrasound (HIFU) and radiofrequency ablation.

The stabilized crosslinked nanobubbles described herein can allow the combination of any of the above noted therapeutic agents and therapies to be administered at a low dose, that is, at a dose lower than has been conventionally used in clinical situations.

A benefit of lowering the dose of the combination therapeutic agents and therapies administered to a subject includes a decrease in the incidence of adverse effects associated with higher dosages. For example, by the lowering the dosage of a chemotherapeutic agent, such as doxorubicin, a reduction in the frequency and the severity of nausea and vomiting will result when compared to that observed at higher dosages. Similar benefits are contemplated for the compounds, compositions, agents and therapies in combination with the nanobubbles.

By lowering the incidence of adverse effects, an improvement in the quality of life of a patient undergoing treatment for cancer is contemplated. Further benefits of lowering the incidence of adverse effects include an improvement in patient compliance, a reduction in the number of hospitalizations needed for the treatment of adverse effects, and a reduction in the administration of analgesic agents needed to treat pain associated with the adverse effects.

It will be appreciated that the stabilized nanobubbles can be used in other applications besides diagnostic, therapeutic, and theranostic applications described above. Nanobubble ultra sound contrast agents have shown great potential in areas of health including cardiovascular and eye diseases, as well as neuromusclular disorders such as Duschenne Muscular dystrophy. Inflammation has been associated with hypoxia. Nanobubbles can deliver oxygen to hypoxic cell and tissues and can be a potential treatment option. In addition, the early stage of atherosclerosis has been manifested with over-expressed intercellular adhesion molecule-1 (ICAM-1), an inflammatory marker. By including ICAM-1 recognizing monoclonal antibodies in the nanobubble membrane or shell, nanobubbles can recognize and adhere to intercellular adhesion molecule-1 (ICAM-1). These types of nanobubble formulations can be used to detect early stages of atherosclerosis, and can be effective in detecting acute cardiac transplant rejection.

Moreover, stabilized nanobubbles described herein can also be used for the treatment of Parkinson's disease. In this regard, the nanobubbles can be used to deliver apomorphine, a particularly beneficial but unstable drug for treating Parkinson's disease, through the blood brain barrier.

Although the use of nanobubbles in medicine is in an early development stage, it is possible that in the future, the applications of nanobubbles in medicine will be as far reaching if not more than that of microbubbles whose applications span across the areas of malignant, infectious, cardiovascular and autoimmune diseases.

The following example is for the purpose of illustration only and is not intended to limit the scope of the claims, which are appended hereto.

### EXAMPLES

### Example 1

This example describes the generation Pluronic-stabilized nanobubbles. These are created by adding interpenetrating crosslinking biodegradable polymer N, N-diethyl acrylamide (NNDEA) and N,N-bis(acryoyl) cystamine (BAC) (FIG. 1) and integrating additional polyethylene glycol (PEG) groups above and beyond those of the Pluronic PEO subunits on the surface of the nanoparticles. Incorporation of crosslinking agents was found to increase stability of Pluronic polymeric micelles below their critical micelle concentration (CMC). In these crosslinked Pluronic-lipid-perfluorocarbon bubbles (CL-PEG-NB) the hydrophobic network is non-covalently integrated into the inner ring or the hydrophobic domain of the bubble, which should improve structural stability while retaining membrane flexibility and reduce diffusion of hydrophobic perfluorocarbon gas out of the core. In this example, the agents were characterized in vitro and their biodistribution and extravasation were examined in a LS174T colorectal tumor xenograft in live mice.

### Materials and Methods:

### Formulation of Nanobubbles

To formulate stabilized CL-PEG-NB, the lipids DPPC (1,2-Dipalmitoyl-sn-Glycero-3-Phosphocholine), DPPA (1,2 Dipalmitoyl-sn-Glycero-3-Phosphate), DPPE (1,2-dipalmitoyl-sn-glycero-3-phosphoethanolamine) (Avanti Polar Lipids, Pelham, Ala.), and mPEG-DSPE (1,2-Distearoyl-phosphatidylethanol amine-methyl-poly ethylene glycol conjugate-2000) (Laysan Lipids, Arab, Ala.) were dissolved in chloroform in a 4:1:1:1 mass ratio. The solvent was then removed by evaporation, leaving a lipid film. The film was hydrated by adding 1 ml of 0.6 mg/ml Pluronic solution (Sigma Aldrich, Milwaukee, Wis.) in 0.5% Irgacure 2959 (Fisher Scientific; Pittsburgh, Pa.) in PBS in the presence of glycerol (50 μl) at 75° C. for 30 min. Next, NNDEA (Polysciences, Warrington, Pa.) and BAC (Sigma Aldrich, Milwaukee, Wis.) (2:1 weight ratio) were added, and air was removed from the sealed vials and replaced with octafluoropropane until the vial pressure equalized. Finally, the vial was shaken on a VialMix shaker (Bristol-Myers Squibb Medical Imaging, Inc., N. Billerica, Mass.) for 45 s, and the bubble vials were irradiated at 254 nm using a UV lamp (Spectronics Co. Westbury, N.Y.) for 30 min. If not used immediately, CL-PEG-NB were stored at 4° C.

To formulate Pluronic PEG-NB, the lipid film was prepared using above lipids and hydrated by adding 1 ml of 0.6 mg/ml Pluronic L10 solution in the presence of glycerol (50 μl). After keeping the solution at 75° C. for 30 min, air was removed from the sealed vials and octafluoropropane (C3H8) was added to the vials until vial pressure equalized. Then, the vial was shaken on a VialMix shaker for 45 s. Bubble samples were stored at 4° C. until use.

### Nanobubble Characterization

The mean diameter and polydispersity of Pluronic nanobubbles were measured using dynamic light scattering (DLS) (90 Plus, Brookhaven Instruments Corp). Measurements were performed at 25° C., with a laser wavelength of 660 nm at an angle of 90° C. Bubble size was measured by diluting a sample 1:1000 with PBS at pH 7.4 (n=3). Bubble size is reported as a number average. Nanobubble morphology was imaged using scanning electron microscopy (SEM) and gated Stimulated Emission Depletion (STED) imaging. In order to prepare samples for SEM imaging, a drop of freshly prepared bubble solution was placed on dust-free foil and kept in a desiccator to evaporate the solvent. Then the samples were sputter coated with palladium and images were obtained using scanning electron microscope (Hitachi S4500) with a gun acceleration voltage of 3.0 kV and 7 mm working distance 15.

The qNano platform from Izon Science (Izon Science, Christchurch, New Zealand) was used to analyze the absolute diameter and the concentration of CL-PEG-NB. The qNano was equipped with the NP150 nanopore for detection of particles in the range of 70-200 nm. The optimized parameters of the qNano were adjusted (45 mm stretch, 0.7 V current) to obtain a stable baseline current with PBS buffer. Then, 40 μl of diluted CL-PEG-NB solution was loaded to the upper fluid cell and pressure (0.5 kPa) applied to obtain the desired 500 particle count. The size and concentration distribution plots were created by Izon Control Suite software, version 2.1.

### In Vitro Ultrasound Stability Characterization

A custom tissue mimicking phantom (ATS Laboratories, Bridgeport, Conn.) was immersed in a 1 L glass beaker containing 700 ml of PBS (Fisher Scientific; Pittsburgh, Pa.) at 37° C. Nanobubble solution (700 μl) was injected into the PBS solution, and the contents of the beaker were continuously stirred at 150 rpm to agitate the bubbles (FIG. 3A). The change in contrast as a function of time was measured using an AplioXG SSA-790A clinical ultrasound imaging system (Toshiba Medical Imaging Systems, Otawara-Shi, Japan) equipped with a 12 MHz linear array transducer. System acquisition parameters were set to Contrast Harmonic Imaging (CHI) with 8.0 MHz harmonic frequency, 0.1 mechanical index (MI), 1 Hz imaging frame rate, 65 dB dynamic range, and 80 dB gain.

Images were obtained over a 60 min period using a low imaging frame rate (0.1 Hz). Regions of interest (ROI) of the same size were drawn in the tissue phantom area and in the contrast agent vicinity. The data were exported to Excel (Microsoft, Redmond, Wash.) and normalized by the contrast of tissue phantom. The signal intensity (logarithmic scale of the normalized data) as a function of time was plotted to obtain the decay rate of nanobubbles. To acquire the rate of nanobubble dissolution over 24 h period, at different time points (t=0, 1, 3, 6, 24 h) of post injection of bubbles, 10 images were obtained and signal intensity was plotted as a function of time.

### Cell Culture

LS174T human colorectal adenocarcinoma cells (ATCC, Manassas, Va.) were cultured in complete MEM medium (10% fetal bovine serum, 1% penicillin-streptomycin; Invitrogen, Carlsbad, Calif.) and placed in a humidified atmosphere at 37° C. and 5% CO₂. At 90% confluence, cells were detached using 0.25% trypsin-EDTA (Invitrogen, Carlsbad, Calif.) for passaging.

### Animal Preparation and Tumor Inoculation

Animals were handled according to a protocol approved by the Institutional Animal Care and Use Committee at Case Western Reserve University and were in accordance with all applicable protocols and guidelines in regards to animal use. In all procedures, the animals were anesthetized with 3% isoflurane with 1 L/min oxygen. LS174T cells (1×10⁷ cells) suspended in 200 μl of growth medium were injected subcutaneously into the flank of each mouse.

### Ultrasound Biodistribution of Nanobubbles In Vivo

Two weeks after inoculation, mice with tumor diameter of 0.8 cm or larger, were selected for biodistribution studies (n=3 per group). To visualize ultrasound images of liver, kidney, and right subcutaneous tumor, the animal was imaged through the sagittal plane. After injection of nanobubbles, the change of tissue contrast of liver, kidney, and tumor was measured using Contrast Harmonic Imaging (CHI, frequency 8.0 MHz; MI, 0.08; dynamic range, 65 dB; gain, 80 dB; imaging frame rate, 1 frame/s). The images were acquired in raw data format as a function of time. Fifteen seconds after raw data acquisition started, nanobubble solution was administrated and continuous image acquisition continued for 8 min. At this point, image acquisition continued for 22 min. with an imaging frame rate of 0.2 frames/s.

The raw data were processed with software provided by the scanner manufacturer. The liver, kidney, and tumor areas were delineated by drawing regions of interest (ROIs) and the signal intensity in each ROI as a function of time (Time Intensity Curve—TIC) was calculated.

The data were exported to Excel, the baseline was subtracted from TIC, and the calculated peak value of TIC was used to normalize the data to obtain the decay of signal at each time point. The log of the data was fitted by simple linear regression to derive the decay slope.

### In Vivo Biodistribution of Nanobubbles Measured Using Fluorescence Molecular Tomography (FMT)

In vivo biodistribution was studied using FMT (FMT 2500, PerkinElmer Inc., Boston, Mass.). One week before FMT imaging, tumor bearing mice were placed on a special diet to reduce gut autofluorescence. Before injection, mice were anesthetized and imaged on the FMT imaging system to rule out auto fluorescence. Animals were divided into 3 groups. For the first group, mice were injected with 200 μl of 1:3 diluted CL-PEG-NB-Vivotag 680 and FMT imaging was performed at multiple time points post-injection (t=5, 15, 30 min and 3 h, n=5). PEG-microbubbles (PEG-MB n=3) were injected as a clinically-relevant control and saline (n=2) was injected as a negative control. FMT was carried out using the 680 nm channel and body scanning 3D reconstructions of the imaging data were performed with TrueQuant software (version 3.1). ROI's were selected for each organ, including tumor, in all 3 imaging planes (X, Y, Z) and total fluorochrome concentration was determined per ROI. At t=3 h animals were euthanized and liver, kidney, heart and tumor were harvested and weighed. Uptake of CL-PEG-NB-Vivo 680 in tumor and other organs will be calculated as the percentage of the injected dose per gram of tissue (% ID/g).

### Histological Analysis

Animals were divided into three groups: CL-PEG-NB (n=5), MB (n=3), and no contrast control (n=2). Mice received either 200 μL of contrast material diluted (1:4) with PBS or PBS alone via the tail vein. Three hours after bubble injection, animals were anesthetized again and Texas red tagged lectin solution (Vector Laboratories, Burlingame, Calif.) (0.1 mL of 1 mg/mL) was inject through a tail vein over a period of 20-30 s. Five minutes after lectin injection, PBS perfusion was performed with 50 ml PBS through left ventricle. Organs (lung, liver, kidney) and tumors were excised and embedded in optimal cutting temperature compound (OCT Sakura Finetek USA, Inc., Torrance, Calif.) and frozen on dry ice. The tissues were cut into 8 μm slices using Leica CM1850 cryostat (Leica, Germany). The fluorescence images were obtained and analyzed using AxioVision V 4.8.1.0, Carl Zeiss software (Thornwood, N.Y.).

### Statistical Analysis

All data are presented as mean±standard deviation, unless otherwise noted. Statistical significance of differences between experimental groups was derived using one-way ANOVA model. Two-tailed unpaired student's t-test with unequal variants were used to determine the significance of the outcome. Data analysis was performed with Microsoft Excel and ANOVA.

### Results

### Nanobubble Characterization

DLS and qNano measurements showed CL-PEG-NB diameters of 95.2±25.2 nm (n=10) and 90±3 nm, respectively (FIGS. 28A-B). Gated Stimulated Emission Depletion (STED) confocal images confirmed the size and spherical shape of CL-PEG-NB (FIG. 2D). Surface morphology and size distribution of CL-PEG-NB were obtained using SEM. Further, SEM images (FIG. 2E) confirmed that the nanobubbles were 100±70 nm sized, spherical and non-aggregated. A donut shape was seen in this formulation but not seen with the original nanobubbles suggesting that crosslinks are located in an annular fashion within the hydrophobic lipid and Pluronic portions surrounding the hydrophobic gas core. Concentration of CL-PEG-NB obtained from qNano was 7×10¹² particles per ml (FIG. 2C), and was 2.5×10¹² particles per ml for the PEG-NB, (data not shown).

### In Vitro Echogenicity Characterization

In vitro stability was analyzed by imaging the nanobubbles at 37° C. under constant agitation and calculating the relative signal loss over time. FIG. 29A shows representative gray scale ultrasound images of PEG-NB and CL-PEG-NB in vitro in the tissue mimicking phantom. The ratios of mean echo-power values of contrast agents (both PEG-NB and the CL-PEG-NB) and the tissue were plotted over time period of 60 min (FIG. 3B inset). The initial contrast at time t=0 was significantly higher with CL-PEG-NB (P<0.01) than the initial contrast of regular PEG-NB. The CL-PEG-NB showed a 2.8 times lower decay rate compared to the decay rate of non-crosslinked PEG-NB (−0.266 db/min compared to −0.742 db/min) over 1 h (FIG. 3 insert). As shown in FIG. 3b the signal intensities of CL-PEG-NB at 6 and 24 h are significantly higher than PEG-NB (P<0.05).

In Vivo Biodistribution of Nanobubbles Visualized with Ultrasound

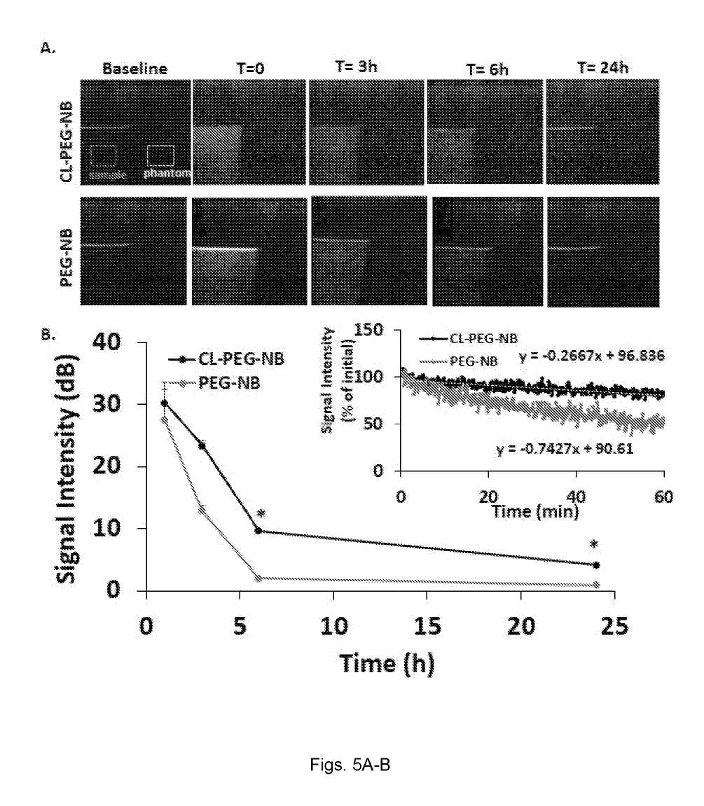

In vivo ultrasound biodistribution of nanobubbles was examined using ultrasound. FIG. 30 shows representative contrast images of selected ROIs (tumor, kidney, liver). The mean echo-power value in the ROIs as a function of time or time-intensity curve (TIC) is shown in FIGS. 5A and 5B. Subsequently administrated of either normal PEG-NB or the CL-PEG-NB via the tail vein of LS174T tumor model bearing mice, the three tested regions (kidney, liver and tumor) showed rapid contrast enhancement. The peak enhancement occurred in both cases 21s after injection, which was followed by gradual wash-out in all tested regions. The PEG-NB accumulation in the kidney and liver was 1.4 times higher than the CL-PEG-NB accumulation in the same organs. CL-PEG-NB showed nearly 2 fold higher enhancement in the tumor area compared to PEG-NB.

After peak enhancement the bubble signal dissipation occurred in two phases. FIG. 31 shows the percent of peak signal intensity of tested regions as a function of time after the peak enhancement that corresponds to the initial phase of bubble dissipation. The decay slopes of CL-PEG-NB in tumor and kidney were significantly slower than those of normal PEG-NB in the initial phase (P<0.05; 15.63±4.06 compared to the 25.03±3.22 in kidney and 13.05±4.19 compared to the 21.96±3.03 in tumor). There were no significant differences observed in the decay slope in the liver during the initial phase.

In Vivo Biodistribution Studies Measured with FMT

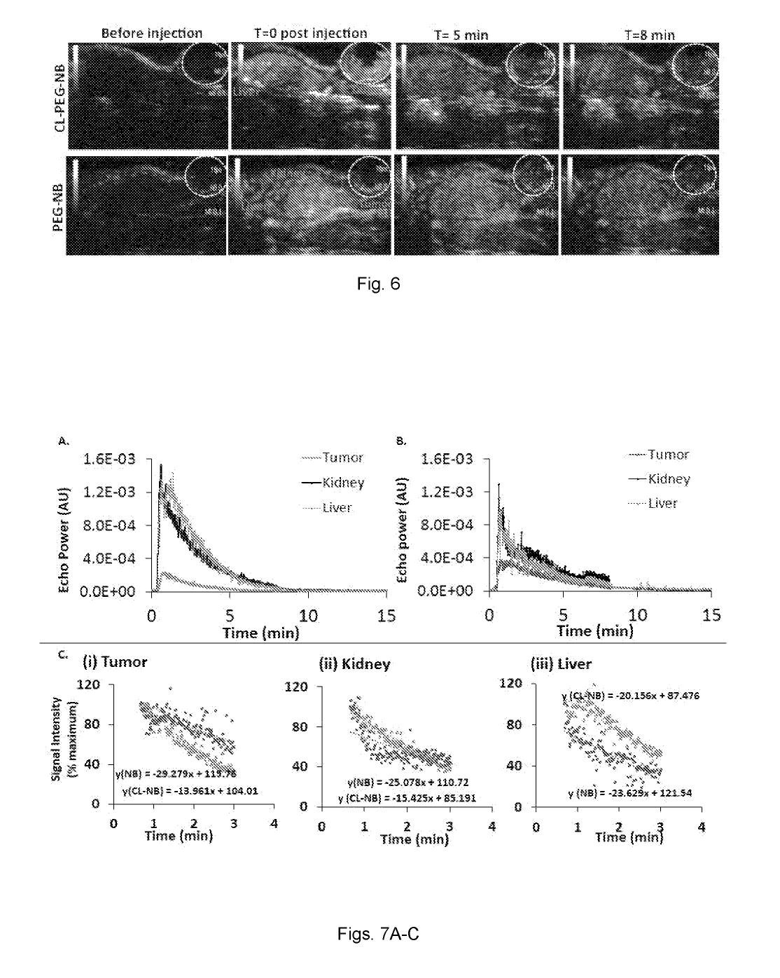

The FMT signal was initially validated using a phantom experiment which showed linear correlation between signal and the various concentrations of bubbles tagged with the fluorescent probe. To quantify the percentage of initial dose of bubbles accumulated per gram of tissue (% ID/g), the fluorescence signal of each organ was analyzed using FMT scan and normalized to the mass of the organ recorded after organ excision. FIG. 6A shows the representative FMT images that were taken 1 h after IV injection of the CL-PEG-NB, PEG-MB and saline control. As shown in FIG. 6B, the intratumoral accumulation of fluorescent signal in tumor from PEG-MB was significantly lower compared to the CL-PEG-NB at each time point post injection (p<0.01).

FIG. 7 shows biodistribution of CL-PEG-NB compared to PEG-MB. We did not observe any statistically significant differences in bubble accumulation in liver immediately after or 1 h post-injection. However, in the kidney, the PEG-MB accumulation was significantly increased in all time points compared to CL-PEG-NB.

### Histological Analysis

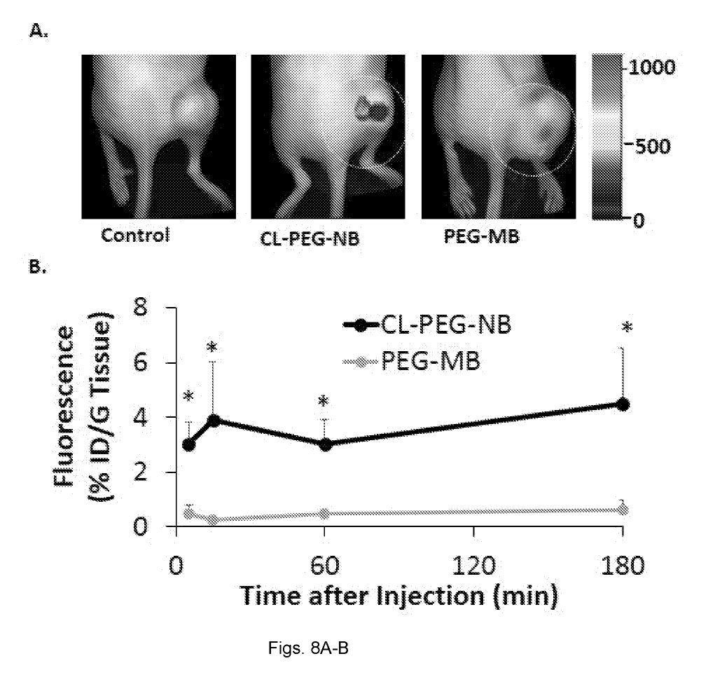

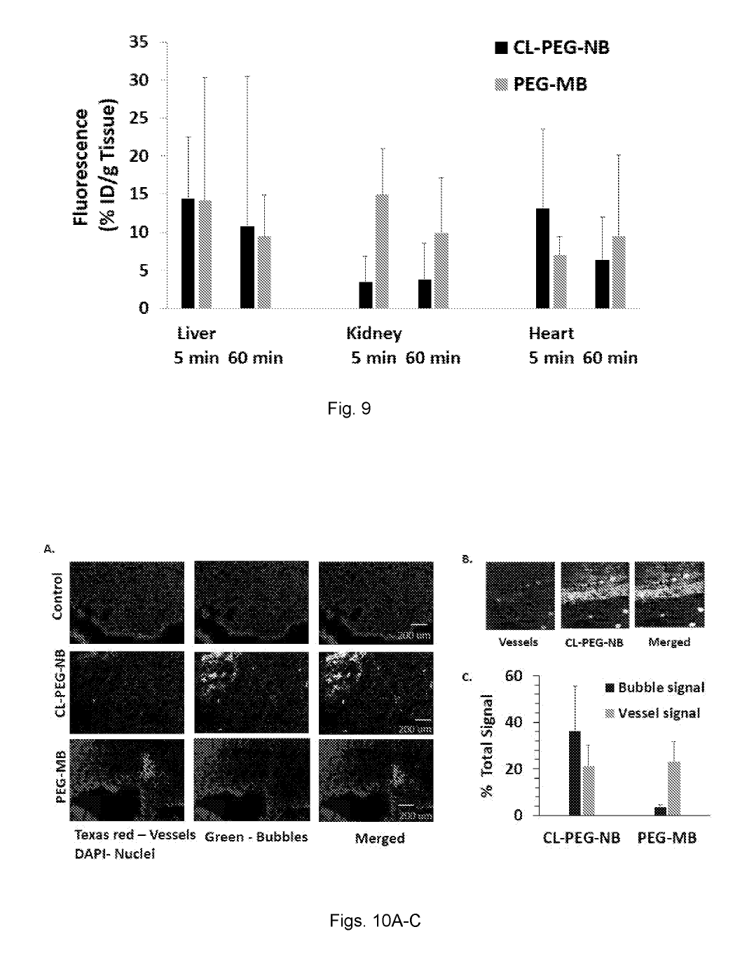

Results are shown in FIG. 8. Here, the red color indicates the vessels stained with Texas red and the green color indicates presence of nanobubbles or nanobubble components. In the CL-PEG-NB group, bubble signal appeared inside the tumor, outside the vessels, which provides strong evidence of bubble extravasation and subsequent interstitial penetration. In the PEG-MB group the bubbles remained primarily within the vasculature (FIG. 8A, 8B). In both cases signal from tumor vasculature appeared to be 21-23% of the total tissue (FIG. 8C). The nanobubble signal intensity in tumor tissue was 12 times higher (36±19% of the total tumor tissue) compared to the microbubble signal (3±9%) and was significantly different (p<0.05).

We formulated a stabilized ultrasound-visible UCA in the size range of nanoparticles typically utilized for molecular imaging and drug delivery in cancer applications. DLS, SEM and STED data provide evidence of the size and morphology of the crosslinker stabilized Pluronic bubbles which are suitable for imaging and delivery applications. Importantly, the small size does not affect the echogenicity of the new formulation. The distinct donut shape seen in SEM images with the CL-PEG-NB formulation suggests stable crosslinks arranged in an annular fashion in the hydrophobic core of the bubble. The persistence of this structure throughout sample preparation also may suggest that these networks play a key role in stabilizing the nanobubble shape. The “deflated” look presumably results from gas diffusion out of the constructs. In vitro echogenicity of regular PEG-NB and the CL-PEG-NB was compared next. Immediately after injection of nanobubbles, the signal was significantly higher (p<0.01) for the CL-PEG-NB, compared to PEG-NB. Based on qNano data, while the concentrations for both formulations were in the 10¹² range, there were indeed more CL-PEG-NB, which could account for the higher signal. It is also possible that higher signal generated by CL-PEG-NB could be due, in part, to more gas entrapped trapped in the high surface area of the crosslinked NNDEA network. The elasticity of the crosslinked polymer also accounted for the enhanced echogenicity. CL-PEG-NB also showed a decreased rate of decay in ultrasound signal compared to PEG-NB. This can be a result of the crosslinked polymer network, which may reduce diffusion of gas from the construct.

In vivo ultrasound biodistribution studies data demonstrate better tumor enhancement with CL-PEG-NB compared to PEG-NB. The enhanced signal is likely due to persistent echogenicity and stability leading to the greater accumulation and retention of CL-PEG-NBs in the tumor. With the CL-PEG-NB, we observed the contrast not only in the tumor periphery, but also inside the tumor matrix, which suggests improved nanobubble penetration into the tumor in contrast to the regular PEG-NB and other reported nanobubbles.

The liver and kidney contrast enhancement was higher with PEG-NB than CL-PEG-NB. In the same organs, PEG-NB had a higher initial decay rate in the liver and kidney compared to the CL-PEG-NB. In contrast, the CL-PEG-NB cleared from the tumor much more slowly than the non-crosslinked NBs, supporting the notion that CL-PEG-NB may accumulate in tumors more extensively than the PEG-NB. In vivo FMT results show that CL-PEG-NB accumulate in the tumor over time at a much greater rate than microbubbles. A limitation of FMT is the difficulty of assigning the ROI for the selected organ in the reconstructed image, especially in the abdomen. The gut signal may overlap with the liver and kidney signals and may account for the inaccuracy and the high standard deviation of the quantitative assessment of the signal. The signal in the kidney after injection of MB was elevated compared to the CL-PEG-NB injected kidney. This may be due to the renal retention of microbubble. Continued investigation into bubble biodistribution is necessary to better understand this behavior.

Accumulation of nanobubbles in tumors relies on extravasation into the tumor parenchyma. Upon histological analysis, CL-PEG-NB showed more extensive extravasation beyond the tumor vessel area, while large PEG-MB showed limited extravasation. However, EPR-driven extravasation depends greatly on the type, stage and morphology of the tumor among many other parameters. Thus these data are only supportive of the process in our LS174T colorectal tumors and additional studies using different tumor models will be necessary to gather more broadly applicable data.

We have demonstrated the development of stable ultrasound-sensitive nanobubbles by incorporating a interpenetrating crosslinked network of N, N-diethylacrylamide and N, N-bis(acryoyl) cystamine into Pluronic-lipid-perfluorocarbon bubbles. The CL-PEG-NB showed enhanced ultrasound signal and lower decay rate compared to PEG-NB without the crosslink network. The new formulation shows promising performance in terms of in vitro and in vivo stability, echogenicity and extravasation into tumor. Future investigation will explore the feasibility of the nanobubbles as a targeted molecular imaging agent for tumor detection and as a carrier vehicle for image guided chemotherapy since the crosslinked acrylamide mesh augments the cargo capacity within the bubble core.

### Example 2

On the Fate of Mesh-Stabilized Lipid Nanobubbles after Destruction with Ultrasound

The dissipation of ultrasound (US) signal from microbubble contrast agents has been linked to their fragmentation, jetting or sonic cracking, leading to a loss of gas. With strong interest in the use of bubbles as drug delivery vehicles, their ultimate fate is of great importance. It has been hypothesized that remnant shells shed into the surround aqueous medium, folding into liposomes or micelles. To investigate these effects, we have applied cryogenic transmission electron microscopy (cryo-EM) to image nanoscale lipid and polymer-stabilized perfluorocarbon gas bubbles before and after their destruction with high intensity US.

### Methods

Polymer-stabilized lipid nanobubbles (NBs) were made by agitation of a lipid solution(DPPC:DPPE:DPPA), Pluronic L10, acrylamide monomers, crosslinker and irgacure 2959 in the presence of C₃F₈ gas, followed by crosslinking under U.V. light. Bubbles were imaged in an agarose mold in PBS using contrast harmonic imaging (Toshiba, 12 MHz, MI 0.1). Flash/replenish (20 cycles) was used to destroy bubbles. Particle size was determined by dynamic light scattering. For cryo-EM, NBs were applied to EM grids (R2/2, 400 mesh; EMS) glow-discharged for 30 sec at 15 mA and imaged on JEOL 2200FS transmission electron microscope with a total electron dose of <100 e−/A².

### Results

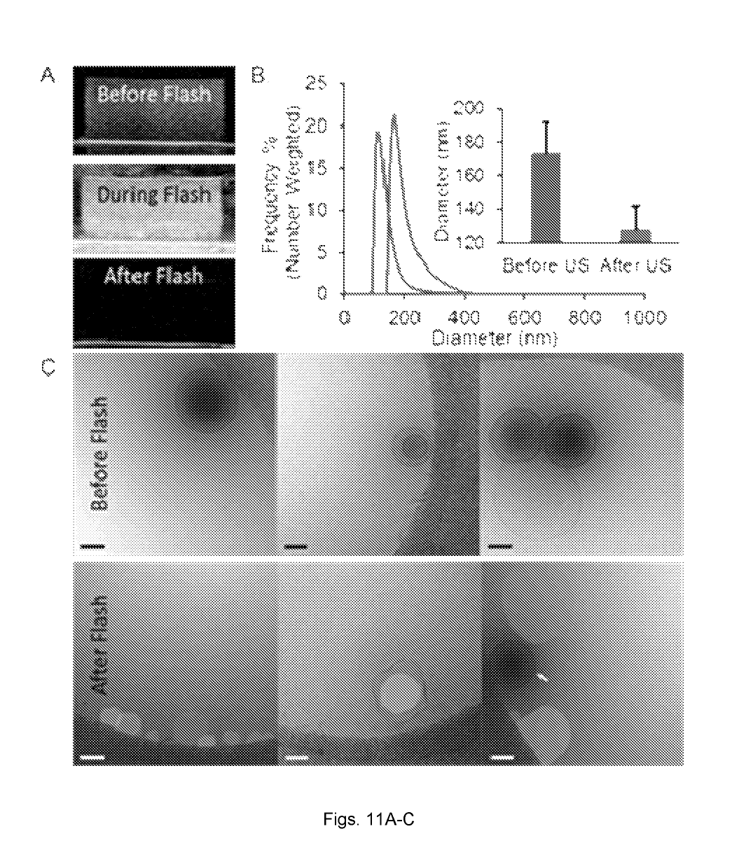

The application of the high intensity US was found to destroy all bubble contrast (FIG. 11a). Mean NB diameter was significantly reduced from 172.7±19.3 nm to 126.7±15.0 nm suggesting a loss of gas from the particles (FIG. 11B). Cryo-EM images of NBs demonstrate that particles have a monolayer shell with a dark center that is likely due to the higher density of the frozen C₃F₈ core relative to the surrounding water layer. Sonicated NBs appeared as amorphous and transparent lipid sheets, indicating a loss of gas (FIG. 11C). While some multi-laminar vesicles were present in the NB solution prior to sonication, none could be visualized in the US disrupted solution. These results suggest that US-disrupted NBs do not reform as liposomes or micelles but rather flatten into round sheets following gas loss. This unexpected result may be due to the hydrophobic acrylamide core helping to maintain particle structure.

### Example 3

### Lipid Acyl Chain Length Improves Stability of Nano-Sized Ultrasound Contrast Agents In Vitro

Ultrasound contrast agents (UCAs), while versatile in their application and composition, require additional optimization with respect to their echogenicity, stability and size to make them suitable for cancer molecular imaging in extravascular applications. We have previously developed lipid and surfactant-stabilized perfluorocarbon gas nanobubbles (NB) capable of extravasating the permeable vasculature of tumors in contrast to conventional microbubbles which are too large (1-10 μm) to enter the interstitial space. The objective of this study was to increase the stability of the NBs by optimization of the lipid hydrophobic chain length to improve the bubble shell in-plane rigidity. Specifically, based on previous microbubble literature, we replaced 1,2-dipalmitoyl-sn-glycero-3-phosphocholine (DPPC), a 16 carbon chain lipid, with 1,2-dibehenoyl-sn-glycero-3-phosphocholine (DBPC), a 22 carbon chain lipid.³

### Materials and Methods

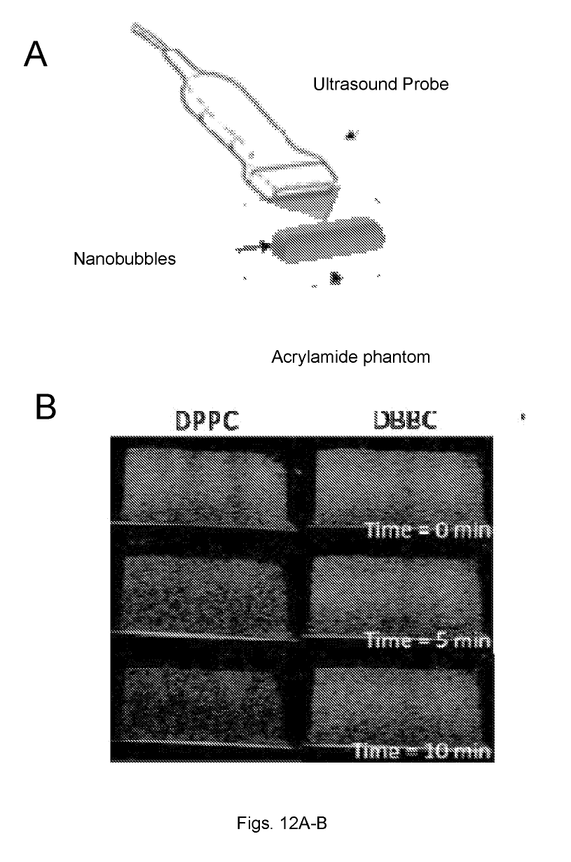

Nanobubbles were made by agitation of lipids, Pluronic L10, and glycerol in the presence of C₃F₈ gas. The mass ratio of lipids in the DPPC containing bubbles was 4:1.5:1:1 mg for DPPC, DPPE, DPPA and PEG respectively per vial of bubbles. The mass ratio of lipids in the DBPC containing bubbles was 6.1:2:1:1 mg per vial of bubbles, where DPPC is completed replaced by DBPC. The bubbles were imaged in an acrylamide phantom (FIG. 12A) in PBS via contrast harmonic imaging (Toshiba, 12 MHz, MI 0.1).

### Results

Increasing the acyl chain length of the most prominent lipid in our formulation significantly improved (p>0.001) the half-life of our nanobubbles from 1.4 minutes to 5.8 minutes (FIGS. 12B-C). This supports what previous literature has found to be true of microbubbles and shows that the same trends are found in nano-sized bubbles. We hypothesize that the longer chains increase lateral cohesion forces, which increases the bending modulus and decreasing lateral density fluctuations of the bubble. The decreased bending modulus lessens buckling of the lipid monolayer, which prevents dissolution. Additional in depth experiments are required to support this hypothesis.

Increasing length of the acyl chain of the most prominent lipid from 16 to 22 carbons, while maintaining about the same molar ratios of all the lipids in the formulation resulted in a 4-fold improvement in half-life of nanobubbles under near continuous US exposure. Future studies will investigate the effects of acyl chain length on bubble stability and accumulation in cancerous tumors in vivo.

### Example 4

### Nanobubble Contrast Agents Enhance Ultrasound Imaging of Prostate Tumors in Mice

Prostate cancer biopsies are increasingly guided by ultrasound (US) imaging, yet poor soft tissue contrast makes delineation between normal/abnormal tissue difficult. There is growing interest in US contrast agents (USCA) to improve differentiation of tumors within the prostate gland and facilitate US-guided biopsies. The most widely used formulations are lipid or protein-stabilized perfluorocarbon (PFC) gas microbubbles (MB) typically exceeding 2 μm in diameter. These bubbles usually show rapid transient tumor enhancement, as they are confined to vasculature. To achieve longer lasting enhancement and improved delineation of tumors, we have developed sub-micron lipid and surfactant-stabilized PFC nanobubbles (NB). Here we compare tumor kinetics of the NBs compared to commercially available MBs.

### Methods

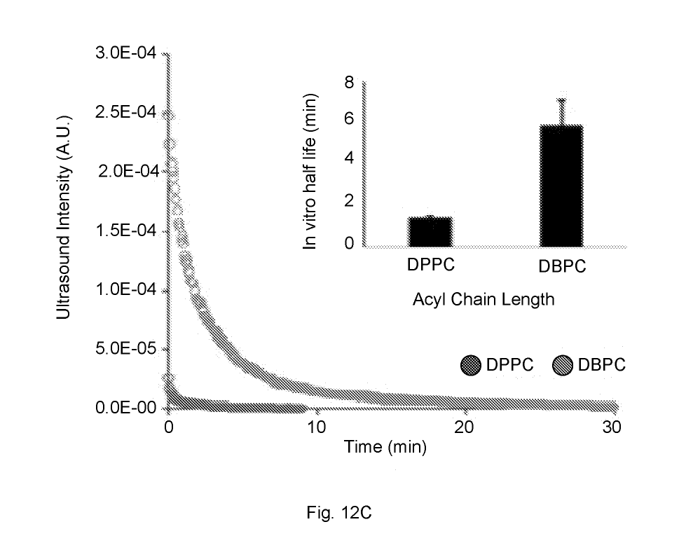

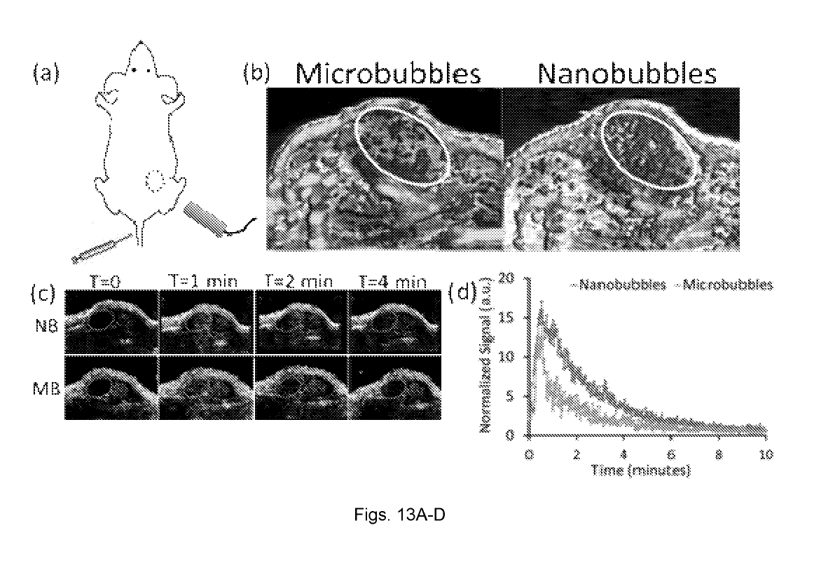

C₃F₈ NBs were formulated by dissolving a cocktail of lipids including DBPC, DSPE-PEG in PBS followed by gas exchange and activation via mechanical agitation. NBs were purified by centrifugation, and size was measured by dynamic light scattering (DLS). Tumors were inoculated in the flank of three male nude mice by injection of PC3 prostate cancer cells in Matrigel, and grown to 5-8 mm (FIG. 13A). Contrast-enhanced US images were acquired with Vevo 3100 (Visualsonics Fujifilm) at lfps, 18 MHz, and 4% power following tail vein injections of 100 ul of either MicroMarker (Visualsonics) or NBs. Maximum intensity projection (MIP) and time-intensity curves (TIC) were obtained in the same mouse for both contrast agents.

### Results

NBs have a diameter of 240±95 nm, (compared to 2-3 μm for MicroMarker). MIP images (FIG. 13B) show that NB provided more signal throughout the tumor cross section compared to MBs at t=15 s. Representative contrast images are shown in FIG. 13C and the mean TIC for all replicates is shown in FIG. 13D. NBs had a half-life of 2.1 min compared to 1 min for microbubbles, and at t=2 min showed a signal intensity nearly 3 times higher than MBs. Higher tumor signal and slower wash out suggests that smaller NBs were able to penetrate out of the leaky tumor vasculature and further into the tumor interstitium. Such NBs may eventually provide a more effective contrast agent compared to MBs and could enhance US guided biopsies.

### Example 5

We developed the second generation nanobubbles which loaded by Doxorubicin. Doxorubicin is most commonly used in clinical as a first-line chemotherapy compound, which shows response in colorectal cancer, lymphoma, ovarian cancer and other malignancies. However, the Doxorubicin has severe toxic side effects, particularly for heart. Based on the EPR effect, the Dox-NB (Doxorubicin-loaded nanobubbles) can entry into the tumor tissue of target through the tumor endothelial gap for explosive release of Doxorubicin triggered by ultrasonic excitation, leading to less systemic side effects and higher locoregional drug concentration in the tumor.

In this example, we show the physical characteristic, ultrasound imaging, and the effect of therapeutic ultrasound in locoregional explosion for Dox-NB. To evaluate the effect of Dox-NB in colon cancer, we next built the tumor xenograft model in nude mice with human colorectal adenocarcinoma cell line LS174T. Then, the distribution of Doxorubicin, the growth of tumor and the overall survival of tumor-bearing mice were measured to identify the physical and biological activity of Dox-NB as a novel drug delivery system.

### Materials and Methods

### Formulation of Dox-NB

To formulate Dox-NB, the lipids DPPC (1,2-Dipalmitoyl-sn-Glycero-3-Phosphocholine), DPPA (1,2 Dipalmitoyl-sn-Glycero-3-Phosphate), DPPE (1,2-dipalmitoyl-sn-glycero-3-phosphoethanolamine) (Avanti Polar Lipids, Pelham, Ala.), and mPEG-DSPE (1,2-Distearoyl-phosphatidylethanol amine-methyl-poly ethylene glycol conjugate-2000) (Laysan Lipids, Arab, Ala.) were dissolved in chloroform in a 4:1:1:1 mass ratio. The solvent was then removed by evaporation and then hydrated by adding 50 ul glycerol and 1 ml 2 mg/ml Doxorubicin, 0.6 mg/ml Pluronic L10 (Sigma Aldrich, Milwaukee, Wis.) and 0.5% Irgacure 2959 (Fisher Scientific; Pittsburgh, Pa.) in PBS solution at 75° C. for 30 min. Next, NNDEA (N, N-diethyl acrylamide) (Polysciences, Warrington, Pa.) and BAC (N, N-bisacryoyl cystamine) (Sigma Aldrich, Milwaukee, Wis.) (2:1 weight ratio) were added, and air was removed from the sealed vials and replaced with octafluoropropane (C₃F₈) until the vial pressure equalized. Finally, the vial was shaken on a VialMix shaker (Bristol-Myers Squibb Medical Imaging, Inc., N. Billerica, Mass.) for 45 s, and the bubble vials were irradiated at 254 nm using a UV lamp (Spectronics Co. Westbury, N.Y.) for 30 min. The bubbles vial then was stored at 4° C. for 1 h and kept the lower clear solution for use.

### Dox-NB Size Analysis and Dox Carrier Amount Measurement

The mean diameter and polydispersity of the Dox-NB were measured using dynamic light scattering (DLS) (90 Plus, Brookhaven Instruments Corp). Measurements were performed at 25° C., with a laser wavelength of 660 nm at an angle of 90° C. Bubble size was measured by diluting a sample 1:1000 with PBS at pH 7.4 (n=3). Bubble size is reported as a number average.

The Dox fluorescence of Dox-NB was read using a TECAN plate reader (infinite M200, San Jose, Calif.) with Excitation and Emission wavelength at 495 nm and 595 nm respectively.

### Optical and Fluorescence Imaging

The Dox-NB were diluted 1000 times with PBS and imaged with a linear probe of 8.0 MHz in a gel mode. The bubbles were imaged again immediately after application of therapeutic ultrasound with an Omnisound 3,000 device (Accelerated Care Plus Corp., Reno, Nev.), at 3 MHz at 2 W/cm2 power density and 20% duty cycle for 1 min.

The 1000 times diluted Dox-NB was dropped onto a slide measuring the morphology of bubbles by confocal microscopy (Zeiss Axio Observer Z1 motorized FL inverted microscope) using a 63× objective.

### Cell Culture

LS174-T human colorectal adenocarcinoma cells (ATCC, Manassas, Va.) were cultured in complete MEM medium (10% fetal bovine serum, 1% penicillin-streptomycin; Invitrogen, Carlsbad, Calif.) and placed in a humidified atmosphere at 37° C. and 5% CO₂. Cells were passaged until they were 90% confluent, then detached with 0.25% trypsin-EDTA (Invitrogen, Carlsbad, Calif.).

### Cell Toxicity Test

One day prior to the treatment, cells were passaged at confluence and plated in flat bottom, clear, cell culture treated 96-well plates (Falcon, Franklin Lakes, N.J.) using a cell density of 2.5×10⁴ cells/ml, 100 ul for each well overnight. After 24 h of incubation, the surface medium were aspirated carefully and cells were treated for 3 h as following groups:
1 No treat Incomplete MEM medium were added.
2. Dox 1 μg/ml Doxorubicin in incomplete MEM medium were added.
3. Dox-NB 2000 times diluted original Dox-NB in incomplete MEM medium were added.
4. Dox+TUS (therapeutic ultrasound) 1 μg/ml Doxorubicin in incomplete MEM medium were added plus therapeutic ultrasound treatment.
5. Dox-NB+TUS 2000 times diluted original Dox-NB in incomplete MEM medium were added plus therapeutic ultrasound treatment.

Plate wells were filled with relevant treatment medium, sealed with Parafilm® M (Fisher Scientific; Pittsburgh, Pa.), then inverted the plate to ensure the cells contact with Dox-NB for 20 min. For plus ultrasound treatment groups, cells were exposed to therapeutic ultrasound with an Omnisound 3,000 device (Accelerated Care Plus Corp., Reno, Nev.), at 3 MHz at 2 W/cm2 power density and 20% duty cycle for 1 min. After 2 h incubation at 37° C. with 5% CO₂ incubator for each treatment group, the solution was removed and washed with incomplete MEM medium 3 times and 200 ul complete MEM medium were added to each well. The cells were again kept in incubation for 3d. After that, 1:10 diluted solution of WST-1 reagent ( ) replaced the medium and incubated for 1 h and then the plate was scanned with a TECAN plate reader (infinite M200, San Jose, Calif.) at 450 nm.

### Measurement of Intracellular Drug Uptake

LS174-T cells were grown in a 12-well cell culture plate at a cell concentration of 2.5×10⁴ cells/ml one day before the treatment. The treatment groups were the same as cell toxicity test above. After 2 h treatment in incubator, the drugs were aspirated and washed with sterile PBS 3 times. The attached cells were fixed with 3% formaldehyde solution for 10 min. After 3 times wash with PBS, the cells of different treatment groups were imaged at 5× and 20× on the Zeiss Axio Observer Z1 motorized FL microscope with the same parameter and scanned zones. Average Dox fluorescence of cells in different treatment groups was calculated by ImageJ.

### Animal Preparation and Tumor Model

Tumor Inoculation Mice were handled according to a protocol approved by the Institutional Animal Care and Use Committee at Case Western Reserve University in accordance with all applicable protocols and guidelines in regards to animal use. Athymic nude mice (NCR nu/nu) were purchased from the Athymic Animal and Xenograft Core Facility of Case Western Reserve University. In all procedures, mice were anesthetized with 3% isoflurane with 11/min oxygen. Then, 200 μl LS-174 T cells (1×10⁶ cells/ml) suspend in incomplete MEM were injected subcutaneously into the flank of each mouse.

### Measurement of Drug Distribution In Vivo

### Treatment Groups

After tumor inoculation, the growth of tumor was measured every other day. When the diameter of tumors was 810 mm in size, the mice were randomly divided into four groups (20 mice/group) as follows: no-treatment group, Dox+TUS group, Dox-NB group and Dox−NB+TUS group. In all procedures, mice were anesthetized with 3% isoflurane with 1 L/min oxygen. Mice in no-treatment group were not given any treatment, and in the other, mice received 100 μg of Doxorubicin in 200 μl PBS as a single i.v. injection. After injection, mice in Dox+TUS group and Dox-NB+TUS group received therapeutic ultrasound-mediated locoregional irradiation for tumor. At 3 h post the treatment, mice were subjected to carbon dioxide-induced euthanasia, and the tumor tissues were picked out for analysis.

### Drug Distribution in Mice Tumors of Homogenize Tissue

Tumor tissues were homogenized in water (10 ml/g) after washing in PBS and weighting. 200 μl of the tissue homogenate was placed in 2 mL microcentrifuge tubes and then 100 ul of buffer, 200 ul of water, and 1,500 μl of acidified isopropanol (0.75 N HCl) were added. The tubes were vortexed to ensure complete mixing, and Doxorubicin was extracted overnight at −20° C. The next day, the tubes were warmed to room temperature, vortexed for 5 minutes, centrifuged at 15,000 g for 20 minutes. The samples were plated into 96-well plate to measure the fluorescence intensity using TECAN fluorometric plate reader, and then the concentration of Dox in tissue was calculated.

Masetro images and Histology Analysis

Half of the ex vivo tumors were imaged using a CRi Maestro fluorescence imaging system. The residual tumors were excised and mounted tissues in optimal cutting temperature compound (OCT Sakura Finetek USA, Inc., Torrance, Calif.) and frozen at −80° C. The tissues were cut into 10 um slices using Leica CM1850 cryostat (Leica, Germany), stained with DAPI using standard techniques. The tissue sections were imaged at 5× and 20× on the Zeiss Axio Observer Z1 motorized FL inverted microscope.

### Long Term In Vivo Treatment Analysis (Tumors Volume Decrease and Survival Test)

Fifteen LS174T-tumor-bearing mice were used in this study. After tumor inoculation, the growth of tumor was measured every other day. When the diameter of tumors was 8˜10 mm in size (day 0), the mice were randomly divided into 3 groups (5 mice/group) as follows: no-treatment group, Dox group and Dox-NB+TUS group. No-treatment mice were not given any treatment. Mice in Dox group received 100 ug of Doxorubicin in 200 ul PBS as a single i.v. injection. Mice in Dox-NB+TUS group received 25 ug of Doxorubicin in 200 μl PBS as a single i.v. injection. At 6 days post the first treatment, the same treatment was repeated. From day 0, tumors were measured every other day by calipers. Tumor measurements were converted to tumor volume (V) using the formula: V=W²×Y/2; where W and Y are the smaller and larger perpendicular diameters, respectively. All the mice were treated and monitored according to a protocol approved by the Institutional Animal Care and Use Committee at Case Western Reserve University in accordance with all applicable protocols and guidelines in regards to animal use. The score was evaluated for each mice with their status every other day. When the score arrived 40, observation was needed every day. By 60 score, the mice were subjected to CO₂-induced euthanasia. Up to 28 days, the experiment was terminated with recording the volume of tumors and survival time of each tumor-bearing mice.

### Statistical Analysis

All data are presented as mean±STDEV (standard deviation) unless otherwise noted. All statistical analyses were performed using SPSS software (Version 19; SPSS Inc. Chicago, Ill., USA). Statistical significance of differences between experimental groups was derived using student's t test or one-way ANOVA test. P<0.05 was considered to be significant.

Results

Dox-NB Characterizations and Drug-Loaded Amount

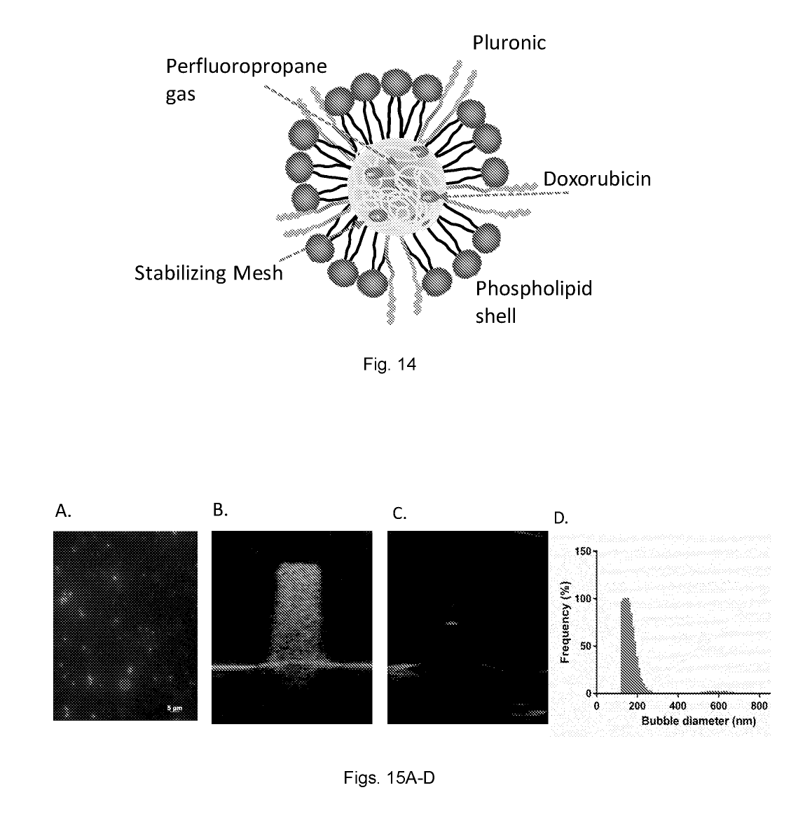

The structural representation of Dox-NB was shown in FIG. 14. After the procedure of Dox-NB formulation, the vial was placed upside down at 4° C. for 1 hour for layering. The bottom layer with clear red liquid is the small size of NB, and the above layer with unclear red liquid was the big size of NB and MB, whereas the upper layer is foam (FIG. 15A). 1000 times dilution of Dox-NB solution was dropped onto slide for measurement by fluorescence microscopy using a 63× objective, by which we could see the nanobubbles with red dot in rhodamine channel (FIG. 15B). Next, we obtained the nanoparticle with the diameters of 100-300 nm (mean value: 171.5 nm) by DSL measurement (FIG. 15C). Fluorescence value of the Dox-NB solution after 400 times dilution was measured by fluorescence spectrophotometer, and the concentration of Dox was calculated using the standard curve, by which the original concentration of Dox solution was 1332.1±96.3 μg/ml. Before TUS explosion, the ultrasound reflection of nanobubbles can be visualized (FIG. 15D), and then the microbubbles echo disappeared after TUS explosion (FIG. 15E).

As a therapeutic nanoparticle of drug delivery system, it should have three features: 1) The nanoparticles should be with the diameters less than 400-800 nm resulting that the particles can entry into tumor cells though the tumor endothelial gap and tumor cell membrane; 2) The drug should be released from nanoparticles by specific excitation at the target site; 3) The nanoparticles should maintain the stable concentration of drug. Here, we showed that the second generation of Dox-loaded nanobubbles were with the diameters of 100-300 nm (mean value: 171.5 nm). Before and after therapeutic ultrasound, the harmonic echo of the nanobubbles was observed and found that the ultrasound excitation-induced echo of Dox-NB disappeared after treatment, proving that the drug can be released from the nanoparticles by ultrasound explosion.

In the present study, 2 mg/ml of original Dox solution was used for nanoparticles synthesis. It has been reported that the fluorescence signal of Dox is affected by time, temperature and light. Therefore, our nanoparticles were produced without light for the whole process; however, the temperature of the solution still got increased during process of lipids hydration and oscillation with adding C₃F₈. We measured the final drug concentration by fluorescence spectrometer, by which the mean of the concentration was 1.3321 mg/ml. Due to particles gathering, the actual value of the detected concentration might be lower, but it should be 1.3321 mg/ml-2 mg/ml. To better compare with control group, we supposed that the quenching of the fluorescence did not take place with the process of the nanobubbles loading, and then Dox group was compared to/Dox+TUS group with equal concentration. In case of positive result, it further defined that the nanoparticles can deliver more drug to target tumor cells. Compared to same dose of Dox or Dox+TUS solution, the nanoparticles-mediated Dox delivery largely enhanced the concentration of locoregional tumor.

### Cell Viability Test and Fluorescence Image of Cell Treatment

1 μg/ml of Dox should be given to the mice in Dox group or Dox+TUS group. However, due to the quenching of the fluorescence, the concentration of original solution in Dox-NB group and Dox-NB+TUS group was 1332.1 μg/ml, and the concentration of Dox with 2000 times dilution became 0.67 μg/ml.

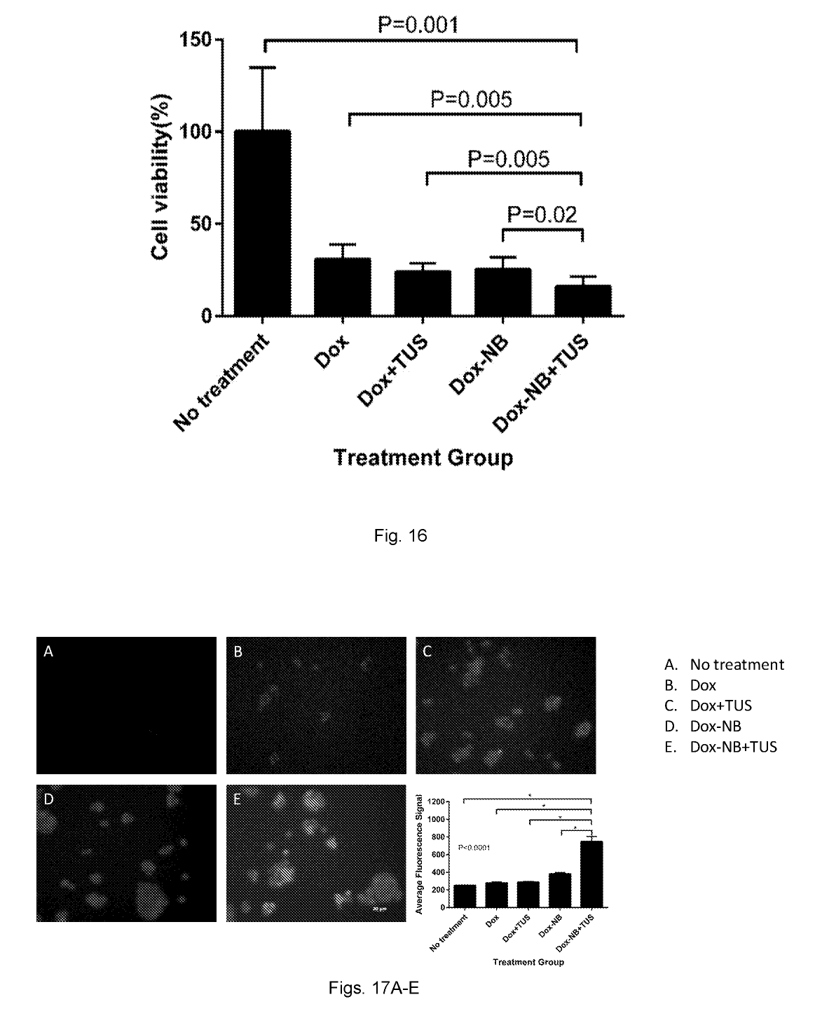

The absorption values without background for each group are listed in Table 1. Although the concentration of Dox in Dox-NB group and Dox-NB+TUS group was lower than that in Dox group and Dox+TUS group, the Dox-NB+TUS still had much more toxic effect in vitro compared to other groups (P<0.05). As shown in FIG. 16, the cell viability of each group was less than 50% compared to control group.

TABLE 1
cell viability test of Dox drugs for LS174T

| Treat groups | Fluorescence value | P (vs. Dox-NB + TUS) |
|---|---|---|
| No treat | 1.19 ± 0.41 | >0.05 |
| Dox | 0.36 ± 0.10 | >0.05 |
| Dox + TUS | 0.28 ± 0.06 | >0.05 |
| Dox-NB | 0.30 ± 0.08 | >0.05 |
| Dox-NB + TUS | 0.19 ± 0.06 | |

The confocal fluorescence microscopy was used to collect four pictures (20×) for each group randomly with Rhodamine channel and 800 ms expose. Then, the value of fluorescence intensity was calculated by ImageJ from 10 cells of each picture randomly (FIG. 17). The value of fluorescence intensity in Dox-NB+TUS group was dramatically increased compared to other groups (P<0.05). Furthermore, the Dox located in both nuclei and cytoplasm with Dox-NB and Dox-NB+TUS treated cells, whereas the Dox mainly located in nuclei with Dox and Dox+TUS treated cells.

Huang and colleagues have investigated the ultrasound-mediated drug delivery using calcein liposomes modified microbubbles and suggested that the calcein can be released from liposomes modified microbubbles with 1 MHz probe and 2 W/cm² sound pressure. It was also reported that Doxorubicin can be released from microparticles for drug delivery. Moreover, Crum and colleagues have shown that microbubbles are destroyed leading to drug release by pulsed ultrasound to the microbubbles with 3.5 MHz, 0.96 MPa for 15 minutes. Taken together, it suggests that ultrasound can be used as an auxiliary tool that helps control drug release. Ultrasound can enhance the anti-tumor efficacy of drug-loaded nanoparticles, and the anti-tumor effects are consisted of ultrasonic cavitation effect, sound effect and other effects. At meanwhile, the radiation ultrasound also improves the cell membrane permeability resulting in much more drug in the tumor cells. In vitro experiment with excitation of ultrasound showed that much more Dox distributed to the tumor cells in Dox-NB+TUS group resulting in more toxic effect to tumor cells compared to other groups.

### In Vivo 3 h Treatment Analysis

Four times dilution of Dox-NB solution was used in vivo, leading that the concentration of Dox in Dox-NB group and Dox-NB-TUS group was 333 μg/ml and the dose for each mice in the two groups was 66.6 μg whereas the dose for each mice in Dox+TUS group was 100 μg.

### Fluorescence Signal In Vivo Analysis of Homogenize Tissue

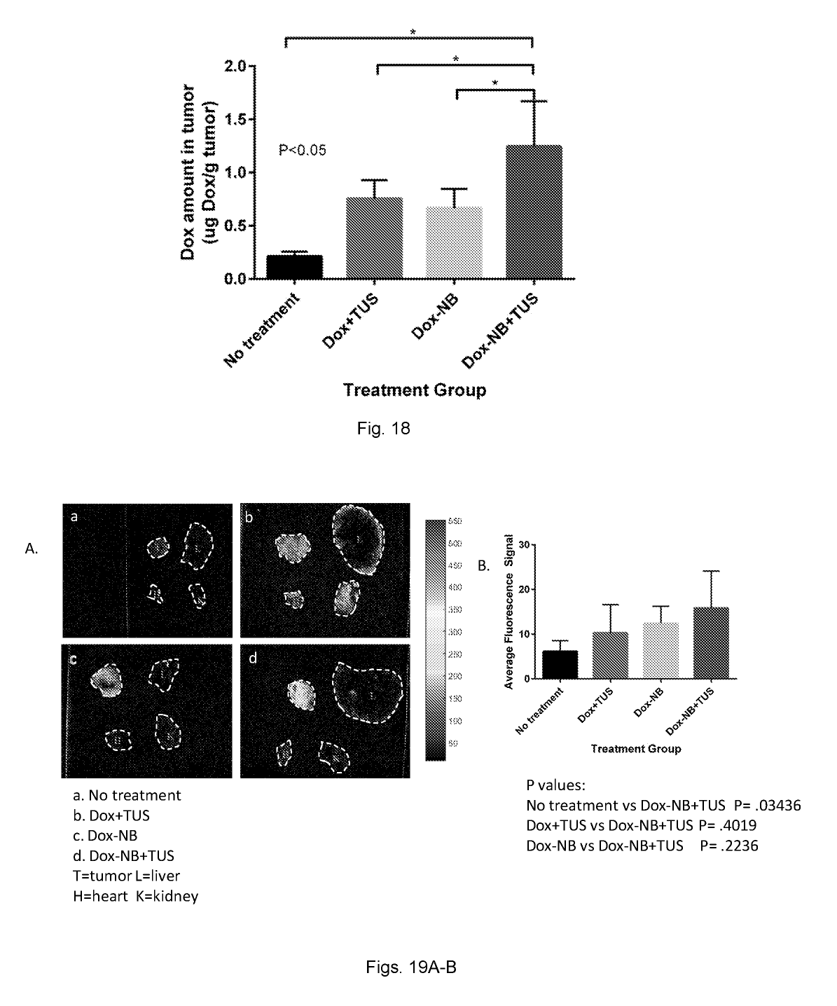

It was showed with tissue homogenates that the concentration of Dox was 1.25 μg per 1 g tumor tissue in Dox-NB+TUS group, which was significantly higher than that in other groups (P<0.05, FIG. 18).

### Masetro Images and Histology Analysis

Tumor tissue, heart, liver and kidney were picked out from tumor-bearing nude mice following washing in PBS, and then pictures were captured by Maestro image system with 1000 ms expose (Extraction, blue filter; Emission, green filter). With the image analysis by unmix pictures, the tumor areas were considered as interest areas to calculate the mean signal value for each group (FIG. 19), showing that the value of fluorescence intensity for the tumor area in Dox-NB+TUS group was higher than other groups but not statistically significant.

### Histology Analysis

Confocal fluorescence microscopy was used for analysis of different fluorescence value for each group with 20× images; the blue color indicated the nuclei stained with DAPI and the red color indicated presence of Dox (FIG. 19). We found that the fluorescence intensity of Dox was stronger in the tumor cells of Dox-NB+TUS group than that of other groups. In order to compare the mean value of fluorescence intensity for each group, ImageJ was used to calculate the value of fluorescence intensity of Dox channel and DAPI channel. The data suggested that the fluorescence ratio (Dox/DAPI) of Dox-NB+TUS group was much higher than that of other groups, pointing that Dox-NB+TUS group had the highest concentration of Dox in tumor cells.

The nanocomposites can be delivered to the tumor tissue through tumor endothelial gap after intravenous injection. It has been documented that locoregional injection of nanoparticles into the tumor has less systemic toxicity; however, the disadvantage of the administration is that the drug can only be dispersed by osmosis. Although some amount of drug could be delivered to the tumor with a little distance via tumor microcirculation, the effect highly depends on the size of the tumor, so locoregional injection is commonly used for radiofrequency and microwave ablation as a supplementary treatment resulting in reduction of locoregional recurrence. The advantages of intravenous administration are that the drug can be delivered to the tumor tissue through the microcirculation and passively overflow from tumor blood vessels for explosive release thereby reducing systemic toxicity with relatively uniform release of drug in the tumor tissue.

In this study, the difference of Dox concentration in tumor tissues at 3 hour post treatment between Dox-NB+TUS group and other groups was analyzed by Maestro-mediated fluorescence imaging for tissue, tissue homogenates, and tissue sections using fluorescence microscopy. Taken these data together, we found that the concentration of Dox was much higher in Dox-NB+TUS group than that in other groups. Indeed, the difference analyzed by tissue homogenates and tissue sections using fluorescence microscopy was statistically significant.

### In Vivo Long Term Treatment Analysis (Tumors Volume Decrease and Survival Test)

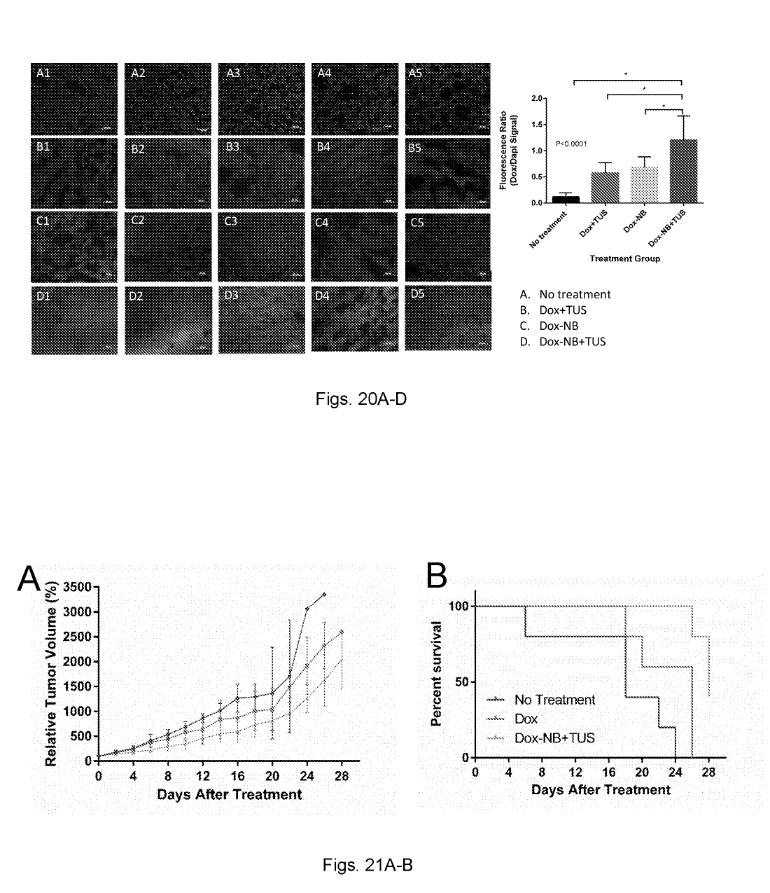

To evaluate the therapeutic effect of Dox-NB+TUS in the colon carcinoma cell line LS174-tumor-bearing nude mice, the size of tumor in each mice was measured for long-term monitoring. Initially, the tumor volume was considered as 100% and then the growth index was calculated and showed in FIG. 20A. Compared to Dox group, Dox-NB+TUS significantly decreased tumor growth (P<0.05). According to the guidelines approved by the Institutional Animal Care and Use Committee at Case Western Reserve University, the scoring criteria were as follows: General Apperance (Normal 0, Mild abnormal 10, Moderately abnormal 20, Severely Compromised 30); Body Condition (Normal to overweight 0, Thin 20, Severe Cachexia 30); Tumor appearance (Non-ulcerated/Not limiting normal mobility 0, Non-ulcerated wound associated with tumor-intact healing or scab present/limiting normal mobility/limiting ability to reach food and/or water 20, Ulcerated or actively bleeding/Preventing mobility, or so cannot eat or drink/limiting ability to breathe normally, or any combination thereof 40); Respiration (Normal rate and effort for species/strain 0, Increased rate and/or effort for species/strain 30, Severe respiratory distress or gasping breathing pattern 60). When the score of mice was higher than 60 or the tumor volume was more than 400 m³, the mice were subjected to euthanasia. Compared to control group and Dox group, Dox-NB+TUS significantly improved the overall survival of the colon tumor-bearing mice using Kaplan-Meier survival analysis (FIG. 20B).

In this example according to clinical treatment, the Dox treatment group or no-treatment group was considered as a control group to confirm that Dox-NB+TUS treatment largely impaired tumor progression with lower tumor volume and longer overall survival compared to other treatments. We also defined that Dox-NB can release high amount of Dox triggering by TUS in the locoregional tumor tissue, and the physical characteristics of ultrasound energy further enhanced the Dox toxicity in tumor cells.

All in all, our experiments also have some limitations. In this example, we considered the initial concentration of Dox as the concentration in Dox-loaded nanobubbles solution, thereby the actual concentration of Dox in Dox-NB solution was much lower than that in the no particles-modified Dox solution like Dox and Dox+TUS solution. Although the actual concentration of Dox is lower, Dox-NB+TUS still releases high amount of Dox in the tumor cells both in vitro and in vivo, further pointing that the novel Dox-loaded nanobubbles are highly specific to release Dox in the tumor tissue for targeting therapy.

While this invention has been particularly shown and described with references to preferred embodiments thereof, it will be understood by those skilled in the art that various changes in form and details may be made therein without departing from the scope of the invention encompassed by the appended claims. All patents, publications and references cited in the foregoing specification are herein incorporated by reference in their entirety.

1. A composition comprising:
a plurality of stabilized crosslinked nanobubbles, each nanobubble having a membrane that defines at least one internal void, which includes at least one gas, the membrane including a plurality of lipids, poloxamers that are incorporated between lipids of the membrane and that are effective to control the size of the nanobubble without compromising in vitro and in vivo echogenicity of the nanobubble, the poloxamers having a molecular weight of 1100 to 3500 Da, and an interpenetrating crosslinked biodegradable polymer that is non-covalently integrated into a hydrophobic inner domain of the nanobubble.

2. (canceled)

3. (canceled)

4. The composition of claim 1, the nanobubbles having an average diameter of about 30 nm to about 300 nm.

5. The composition of claim 1, the nanobubble having an average diameter of about 50 nm to about 200 nm.

6. The composition of claim 1, the interpenetrating crosslinked biodegradable polymer comprising a crosslinked acrylamide polymer.

7. The composition of claim 6, the crosslinked acrylamide polymer comprising the reaction product of N, N-diethyl acrylamide (NNDEA) and N, N-bis(acryoyl) cystamine (BAC).

8. The composition of claim 1, wherein the at least one lipid comprises a mixture of at least two of 1,2-Dipalmitoyl-sn-Glycero-3-Phosphocholine (DPPC), 1,2 Dipalmitoyl-sn-Glycero-3-Phosphate (DPPA), 1,2-dipalmitoyl-sn-glycero-3-phosphoethanolamine (DPPE) 1,2-Distearoyl-phosphatidylethanol amine-methyl (DSPE), 1,2-Distearoyl-phosphatidylethanol amine-methyl-poly ethylene glycol conjugate (DSPE-PEG), or 1,2-dibehenoyl-sn-glycero-3-phosphocholine (DBPC).

9. The composition of claim 1, further comprising at least one targeting moiety that is linked to the membrane.

10. The composition of claim 9, the targeting moiety being selected from the group consisting of polypeptides, polynucleotides, small molecules, elemental compounds, antibodies, and antibody fragments.

11. The composition of claim 1, further comprising at least one therapeutic agent that is contained within the membrane or conjugated to the membrane.

12. The composition of claim 11, the therapeutic agent further comprising at least one chemotherapeutic agent, anti-proliferative agent, biocidal agent, biostatic agent, or anti-microbial agent.

13. A method for imaging a region of interest (ROI) in a subject, the method comprising the steps of:
administering to the subject a plurality of stabilized crosslinked nanobubbles, each nanobubble having a membrane that defines at least one internal void, which includes at least one gas, the membrane including a plurality of lipids, poloxamers that are incorporated between lipids of the membrane and that are effective to control the size of the nanobubble without compromising in vitro and in vivo echogenicity of the nanobubble, the poloxamers having a molecular weight of 1100 to 3500 Da, and an interpenetrating crosslinked biodegradable polymer that is non-covalently integrated into a hydrophobic inner domain of the nanobubble; and
generating at least one image of the ROI by ultrasound imaging the nanobubbles in the ROI.

14. The method of claim 13, the step of administering the composition to the subject further comprising the step of generating at least one baseline image of the ROI prior to administering the composition.

15. (canceled)

16. (canceled)

17. The method of claim 13, the nanobubbles having an average diameter of about 30 nm to about 300 nm.

18. The method of claim 13, the nanobubbles having an average diameter of about 50 nm to about 200 nm.

19. The method of claim 13, the interpenetrating crosslinked biodegradable polymer comprising a crosslinked acrylamide polymer.

20. The method of claim 19, the crosslinked acrylamide polymer comprising the reaction product of N, N-diethyl acrylamide (NNDEA) and N, N-bis(acryoyl) cystamine (BAC).

21. The method of claim 13, wherein the at least one lipid comprises a mixture of at least two of 1,2-Dipalmitoyl-sn-Glycero-3-Phosphocholine (DPPC), 1,2 Dipalmitoyl-sn-Glycero-3-Phosphate (DPPA), 1,2-dipalmitoyl-sn-glycero-3-phosphoethanolamine (DPPE) 1,2-Distearoyl-phosphatidylethanol amine-methyl (DSPE), 1,2-Distearoyl-phosphatidylethanol amine-methyl-poly ethylene glycol conjugate (DSPE-PEG), or 1,2-dibehenoyl-sn-glycero-3-phosphocholine (DBPC).

22. The method of claim 13, further comprising at least one targeting moiety that is linked to the membrane.

23. The method of claim 22, the targeting moiety being selected from the group consisting of polypeptides, polynucleotides, small molecules, elemental compounds, antibodies, and antibody fragments.

24. The method of claim 13, further comprising at least one therapeutic agent that is contained within the membrane or conjugated to the membrane.

25. The composition of claim 24, the therapeutic agent further comprising at least one chemotherapeutic agent, anti-proliferative agent, biocidal agent, biostatic agent, or anti-microbial agent.

26-39. (canceled)
